(19) United States
(12) Patent Application Publication
(10) Pub. No.: US 2004/0191761 A1
(43) Pub. Date: Sep. 30, 2004

(54) Modified adenoviral E1A constructs and methods of use thereof

(76) Inventor: John M. Routes, Centennial, CO (US)

Correspondence Address: SHERIDAN ROSS PC, 1560 BROADWAY, SUITE 1200, DENVER, CO, 80202

(21) Appl. No.: 10/402,339

(22) Filed: Mar. 27, 2003

(51) Int. Cl. C12Q 1/70; C07H 21/02; C12N 7/00; C12N 15/861

(52) U.S. Cl. 435/5; 435/69.3; 435/235.1; 435/456; 435/325; 530/350; 536/23.72

(57) ABSTRACT

Recombinant nucleic acid molecules encoding a mutated Group C adenoviral E1A protein and an antigen are disclosed, as well as mutated E1A-antigen fusion proteins produced by such constructs, and compositions comprising such fusion proteins and individual proteins produced by the recombinant constructs. Also included are modified adenoviral vectors and therapeutic uses for the nucleic acids, proteins, compositions, and viral vectors of the invention.

### GOVERNMENT SUPPORT

This invention was supported in part with funding provided by NIH Grant No. CA7649, awarded by the National Institutes of Health. The government may have certain rights to this invention.

### FIELD OF THE INVENTION

This invention generally relates to recombinant nucleic acid molecules encoding a modified Group C adenoviral E1A protein and an antigen. The invention also relates to modified E1A-antigen fusion proteins produced by such constructs, as well as compositions comprising modified E1A proteins and antigens produced by such constructs. Also included are modified adenoviral vectors and therapeutic uses for the nucleic acids, proteins, compositions, and viral vectors of the invention.

### BACKGROUND OF THE INVENTION

For well over three decades, it has been known that small DNA tumor viruses such as adenoviruses (Ad), papillomaviruses and papova viruses can transform mammalian cells and are oncogenic in a large number of diverse species. The importance of these viruses in human malignancies is now well established. These viruses share some common features. First, they are able to transform a wide variety of mammalian cells, from rodent cells to human cells. Second, the transformation event is characterized by viral integration into the host genome, and the expression of only one or two viral oncogenes is consistently found. Third, although these viruses are evolutionarily distinct, they transform cells by common molecular mechanisms through the expression of functionally and/or structurally similar viral oncoproteins. Fourth, viral transformation results in an inability of these viruses to replicate, and hence, the transformed cells are virion free.

Despite substantial progress in elucidating the molecular mechanisms that the small DNA tumor viruses utilize to transform mammalian cells, a fundamental question remains: why do these viruses so dramatically differ in their oncogenicity, even in species that serve as the natural host for virus infection? Studies comparing oncogenic and nononcogenic adenoviruses in rodents have provided some clues. Results from these studies show that: 1) oncogenic (group A, Ad serotype 12; Ad12) and nononcogenic (group C, Ad serotypes 2 and 5 Ad2/5) adenoviruses transform cells with equivalent efficiency; 2) construction of chimeric Ad5-Ad12 viruses show that the differences in oncogenicity in large part maps to differences between the E1A genes of the two viruses (the E1A oncogene is the primary immortalizing gene in adenovirus); 3) cells transformed by nononcogenic adenoviruses are conditionally oncogenic (i.e., Ad2/5-transformed cells are oncogenic in hosts with impaired innate immunity (NK cell depletion) and impaired acquired cellular immunity (thymectomy); and 4) expression of E1A from nononcogenic and oncogenic adenoviruses in virally transformed cells elicits dramatically different antitumor immune responses.

In contrast to the E1A proteins from oncogenic adenoviruses, expression of non-oncogenic E1A in virally transformed cells elicits a robust innate (NK cells and macrophages) and acquired (CTL) anti-tumor immune response. In addition, there is a striking inverse correlation between the oncogenicity of virally transformed cells and their capacity to be killed by components of the innate immune system. Transformed cells that are nononcogenic are sensitive to killing by NK cells and macrophages, whereas highly oncogenic cells are resistant to killing by both cell types. For example, studies performed by the present inventor have shown that E7-expressing cells are resistant to rejection by animals with competent NK cell and T cell responses, and conversely, E1A-expressing tumor cells were sensitive to NK lysis in vitro and triggered a protective NK and T cell immune response in vivo. These data are summarized in Routes et al., Virology: 277:48-57, 2000. Therefore, while the reasons for disparity in the oncogenicities of small DNA tumor viruses is still in large part unknown, it is clear that the competence of the cellular immune response to clear virally transformed cells is a major factor.

A fundamental question in tumor immunology relates to why tumor-specific antigens differ in their capacity to induce a tumor-specific immune response (TSIR). Many tumors express antigens that are exclusively or preferentially expressed in tumors, but not in normal tissues. However, despite the presence of these potentially immunogenic proteins, tumor-specific immune responses are generally weak in patients with tumors. For example, E7 and E6 oncoproteins are expressed in the tumor cells of over 95% of cervical cancers. Despite the expression of these foreign oncoproteins, tumor-specific immun responses to these oncoproteins are weak and ineffective. The development of immunotherapeutic approaches to bolster tumor-specific immun responses is a major focus in tumor immunology today. The question of tumor antigen immunogenicity is particularly relevant when considering immune responses to virally transformed cells, where the viral oncoproteins themselves have biological activities that dramatically alter the transcriptional machinery of cells and interrupt cell-growth regulatory pathways. Through these biological activities, viral oncoproteins may influence immunogenicity in ways that are distinct from their intrinsic antigenicities. Understanding the molecular basis for differences in viral oncoprotein immunogenicity is critical given the ever-expanding number of transforming viruses that have been implicated in human malignancies. Furthermore, such an understanding will provide insight regarding factors that generally influence the immunogenicity of tumor antigens.

A variety of approaches has been used to enhance the immunogenicity of antigenic, but poorly immunogenic, tumor antigens. Although progress has been made, there has been very limited success in the actual treatment of human malignancies to date. An approach is needed that will enhance the immunogenicity of any tumor antigen, irrespective of the tissue derivation of the cancer or the MHC genotype of the patient. Such a strategy could also be applied to other weakly immunogenic antigens for the treatment of other diseases.

Adenovirus serotype 2 or serotype 5 E1A genes for cancer immunotherapy exhibit many of these desirable characteristics. The introduction of E1A into tumor cells leads to a dramatic decrease in tumorigenicity in a wide spectrum of tumor types and species of animals. Thus, the benefit of E1A in the reduction of tumorigenicity is independent of tumor type or MHC genotype. This ability of E1A to inhibit tumorigenesis has led to the use of E1A in phase I/II clinical trials.

Recognizing the association of the adenovirus E1A protein with viral anti-oncogenicity, various investigators have attempted to manipulate the E1A protein in order to provide a product or composition for reducing tumor growth. For example, Frisch et al. (see, e.g., U.S. Pat. Nos. 5,866,550; 5,516,631; and 5,776,743) describes the use of E1A and amino acid sequences having the biological activity of the E1A gene product thereof to reduce tumor growth.

Hung et al. (see, e.g., U.S. Pat. Nos. 6,197,754; 6,326,356; 5,814,315) have also described the use of E1A and a “mini-E1A” for reducing tumor growth. The mini-E1A of Hung et al. includes an E1A that lacks at least some part of the N-terminus (amino acids 1-119), and preferably includes at least a portion of the C-terminus of E1A, but can exclude amino acids 81-188. Hung et al. disclose that the C-terminal portions of E1A (at least amino acids that are C-terminal of position 211, and preferably amino acids that are C-terminal of position 188) are necessary for the anti-tumor effect via repression of neu expression. Hung et al. further teach that portions of the CR1 region (e.g., 48-60 or 70-80) are not required for the anti-tumor effect via repression of neu expression.

Other investigators have produced recombinant adenoviruses for therapeutic purposes. For example, Ertl et al. (see, e.g., U.S. Pat. Nos. 5,698,202; 6,287,571; 6,019,978), disclose recombinant adenovirus having a deletion of E1 and at least a partial deletion of E3, wherein a rabies virus antigen-encoding cassette is introduced into E1. U.S. Application Publication No. 2002/0037274 to Williams et al. is directed to replication competent adenoviral mutants having a mutation in the E1A RB family member binding site and particularly in the E1A CR2 region which are identified as useful for killing tumors via the anti-tumor effect of E1A. Williams et al. also describe methods for making the virus less immunogenic. PCT Publication No. WO 01/36650 to Onyx Pharmaceuticals, Inc. describes an E1A vector that uses a different promoter and which can have a mutation in the CR2 region of E1A. The vector is used for its anti-tumor growth effects. PCT Publication No. WO 01/72341 to Calydon, Inc. describes an adenoviral vector which is modified and used in conjunction with an anti-tumor agent and/or radiation. The vector can include E1A and/or E1B under the control of different transcription control sequences. A related PCT Publication, WO 01/73093, is directed to adenoviral vectors that use internal ribosome binding sites to direct the expression of genes in the vector. PCT Publication No. WO 01/04282 to Saint Louis University describes replication competent adenoviral vectors which overexpress an adenovirus death protein and which are used for promoting the death of neoplastic cells. The vector can lack the E3 proteins and can include other modifications.

Unfortunately, despite the many advantages of E1A, there is no evidence that the introduction of E1A into tumor cells leads to the induction of tumor-specific immune responses. In fact, observations from the present inventor's own laboratory show that expression of E1A in tumor cells that express tumor antigens fails to induce a measurable, protective tumor-specific immune response. Therefore, there remains a need in the art for an understanding of the relationship between human malignancies and transforming viruses, as well as factors that generally influence the immunogenicity of tumor antigens, in order to develop better therapeutic strategies for treating tumors and other diseases where limited immunogenicity of foreign antigens is an issue.

### SUMMARY OF THE INVENTION

One embodiment of the present invention relates to an isolated nucleic acid molecule, comprising: (a) a first nucleic acid sequence encoding an antigen; and (b) a second nucleic acid sequence encoding a mutated Group C adenoviral E1A protein. The amino acid sequence of the mutated E1A protein differs from the amino acid sequence of a wild-type E1A protein by at least one mutation, wherein the mutation comprises a mutation in the CR2 region of E1A that abolishes the ability of the mutated E1A protein to bind to pRb. The mutated E1A protein retains an amino acid sequence from the wild-type E1A protein that binds to p300/CBP. In addition, the mutated E1A protein retains an amino acid sequence from the wild-type E1A protein that binds to the C-terminal binding protein (CtBP). In one aspect, the amino acid sequence of the mutated E1A protein further differs from the amino acid sequence of the wild-type E1A protein by a deletion of from 1 to about 5 amino acid residues from the C-terminal end of the wild-type E1A amino acid sequence, wherein the mutated E1A protein has anti-oncogenic activity and is immunogenic. Preferably, the mutated E1A sensitizes cells containing the mutated E1A to killing by immune effector cells and/or enhances upregulation of Hsp70 by cells containing the mutated E1A. In one aspect, the antigen in the mutated E1A construct of the invention is selected from: a tumor antigen, a viral antigen, a bacterial antigen, a fungal antigen and a parasitic antigen.

In another aspect, the mutated E1A protein differs from the wild-type E1A protein by a deletion of amino acids in the CR2 region that abolishes the ability of the mutated E1A protein to bind to pRb. In another aspect, the mutated E1A protein differs from the wild-type E1A protein by a substitution or insertion of amino acids in the CR2 region that abolishes the ability of the mutated E1A protein to bind to pRb.

In one aspect, the mutated E1A protein is identical to the wild-type E1A protein by retention of amino acid residues in the CR1 region and amino acid residues in the first 10 residues from the N-terminus of the protein, wherein the retained residues bind to p300/CBP. In another aspect, the mutated E1A protein is identical to the wild-type E1A protein by retention of amino acid residues in the first 60 residues from the N-terminus of the protein, wherein the retained residues bind to p300/CBP.

In yet another aspect, the mutated E1A protein is identical to the wild-type E1A protein by retention of amino acid residues in the CR3 region. In another aspect, the mutated E1A protein is identical to the wild-type E1A protein by retention of amino acid residues in the last 44 amino acid positions from the C-terminus.

In one aspect, the wild-type E1A to which the mutated E1A is compared or from which it is derived is selected from: an adenovirus serotype 5 13S E1A protein represented by SEQ ID NO:2, an adenovirus serotype 2 13S E1A protein represented by SEQ ID NO:6, an adenovirus serotype 5 12S E1A protein represented by SEQ ID NO:4, and an adenovirus serotype 2 12S E1A protein represented by SEQ ID NO:8. In one aspect, the amino acid sequence of the mutated E1A protein further differs from the amino acid sequence of the wild-type E1A protein by a deletion or substitution of at least one amino acid residue, at least about 10 amino acid residues, or at least about 20 amino acid residues, within the amino acid sequence between: (a) about position 189 and about position 270 of SEQ ID NO:2; (b) about position 189 and about position 270 of SEQ ID NO:6; (c) about position 133 and about position 224 of SEQ ID NO:4; or (d) about position 133 and about position 224 of SEQ ID NO:8. In this aspect, the mutated E1A protein has anti-oncogenic activity and is immunogenic. In another aspect, the amino acid sequence of the mutated E1A protein further differs from the amino acid sequence of the wild-type E1A protein by a deletion or substitution of at least one amino acid residue, at least about 10 amino acid residues, or at least about 20 amino acid residues, within the amino acid sequence between: (a) about position 189 and about position 245 of SEQ ID NO:2; (b) about position 189 and about position 245 of SEQ ID NO:6; (c) about position 133 and about position 199 of SEQ ID NO:4; or (d) about position 133 and about position 199 of SEQ ID NO:8. In this aspect, the mutated E1A protein has anti-oncogenic activity and is immunogenic.

In one aspect, the amino acid sequence of the mutated E1A protein is identical to the amino acid sequence of the wild-type protein by retention of amino acid positions: (a) from about 143 to about 188 of SEQ ID NO:2; or (b) from about 143 to about 188 of SEQ ID NO:6. In another aspect, the amino acid sequence of the mutated E1A protein differs from the amino acid sequence of the wild-type E1A protein by a mutation in at least amino acid positions: (a) 124-127 of SEQ ID NO:2; (b) 124-127 of SEQ ID NO:6; (c) 124-127 of SEQ ID NO:4; or (d) 124-127 of SEQ ID NO:8. Preferably, the mutation is sufficient to abolish the ability of the mutated E1A protein to bind to pRb. In one aspect, such a mutation is a deletion of the amino acid positions.

In yet another aspect, the amino acid sequence of the mutated E1A protein differs from the amino acid sequence of the wild-type E1A protein by a mutation in at least amino acid positions: (a) 120-140 of SEQ ID NO:2; (b) 120-140 of SEQ ID NO:6; (c) 120-132 of SEQ ID NO:4; or (d) 120-132 of SEQ ID NO: 8, wherein the mutation is sufficient to abolish the ability of the mutated E1A protein to bind to pRb. In one aspect, such a mutation is a deletion of the amino acid positions.

In yet another aspect, the amino acid sequence of the mutated E1A protein is identical to SEQ ID NO:2, SEQ ID NO:6, SEQ ID NO:4 or SEQ ID NO:8 by retention of amino acids in at least positions 1-10 and from about positions 48 to about position 60, and the mutated E1A binds to p300/CBP. In another aspect, the amino acid sequence of the mutated E1A protein is identical to SEQ ID NO:2, SEQ ID NO:6, SEQ ID NO:4 or SEQ ID NO:8 by retention of amino acids in at least positions 1-40 and from about positions 48 to about position 60, and the mutated E1A binds to p300/CBP. In yet another aspect, the amino acid sequence of the mutated E1A protein is identical to SEQ ID NO:2, SEQ ID NO:6, SEQ ID NO:4 or SEQ ID NO:8 by retention of amino acids in at least positions 1-60, and the mutated E1A binds to p300/CBP.

In one aspect, the mutated E1A comprises, in consecutive order, amino acid positions 1 to about 140 and amino acid positions from about 271 to about 284 of SEQ ID NO:2 or SEQ ID NO:6, wherein amino acids in the CR2 region have been mutated to abolish the ability of the mutated E1A to bind to pRb. In another aspect, the mutated E1A comprises, in consecutive order, amino acid positions 1 to about 188 and amino acid positions from about 271 to about 284 of SEQ ID NO:2 or SEQ ID NO:6, wherein amino acids in the CR2 region have been mutated to abolish the ability of the mutated E1A to bind to pRb. In another aspect, the mutated E1A comprises, in consecutive order, amino acids 1 to about 123, amino acids from about 128 to about 140, and amino acids from about 271 to about 284, of SEQ ID NO:2 or SEQ ID NO:6. In yet another aspect, the mutated E1A comprises, in consecutive order, amino acids 1 to about 123, amino acids from about 128 to about 188, and amino acids from about 271 to about 284, of SEQ ID NO:2 or SEQ ID NO:6. In one aspect, the mutated E1A comprises, in consecutive order, amino acid positions I to about 140 and amino acid positions from about 246 to about 289 of SEQ ID NO:2 or SEQ ID NO:6, wherein amino acids in the CR2 region have been mutated to abolish the ability of the mutated E1A to bind to pRb. In yet another aspect, the mutated E1A comprises, in consecutive order, amino acid positions 1 to about 188 and amino acid positions from about 246 to about 289 of SEQ ID NO:2 or SEQ ID NO:6, wherein amino acids in the CR2 region have been mutated to abolish the ability of the mutated E1A to bind to pRb. In yet another aspect, the mutated E1A comprises, in consecutive order, amino acids 1 to about 123, amino acids from about 128 to about 140, and amino acids from about 246 to about 289, of SEQ ID NO:2 or SEQ ID NO:6. In another aspect, the mutated E1A comprises, in consecutive order, amino acids 1 to about 123, amino acids from about 128 to about 188, and amino acids from about 246 to about 289, of SEQ ID NO:2 or SEQ ID NO:6. In another aspect, the mutated E1A consists of, in consecutive order, amino acids 1 to about 123, amino acids from about 128 to about 188, and amino acids from about 246 to about 289, of SEQ ID NO:2 or SEQ ID NO:6. In another aspect, the mutated E1A comprises, in consecutive order, amino acids 1 to about 132, and amino acids from about 225-238 of SEQ ID NO:4 or SEQ ID NO:8, wherein amino acids in the CR2 region have been mutated to abolish the ability of the mutated E1A to bind to pRb. In another aspect, the mutated E1A comprises, in consecutive order, amino acids 1 to about 132, and amino acids from about 200-243 of SEQ ID NO:4 or SEQ ID NO:8, wherein amino acids in the CR2 region have been mutated to abolish the ability of the mutated E1A to bind to pRb. In another aspect, the mutated E1A comprises, in consecutive order, amino acids 1 to about 123, amino acids from about 128 to about 132, and amino acids from about 225-238 of SEQ ID NO:4 or SEQ ID NO:8. In another aspect, the mutated E1A comprises, in consecutive order, amino acids 1 to about 123, amino acids from about 128 to about 132, and amino acids from about 200-243 of SEQ ID NO:4 or SEQ ID NO:8. In yet another aspect, the mutated E1A consists of in consecutive order, amino acids 1 to about 123, amino acids from about 128 to about 132, and amino acids from about 200-243 of SEQ ID NO:4 or SEQ ID NO:8.

Another embodiment of the present invention relates to a recombinant nucleic acid molecule, comprising any of the above-referenced isolated nucleic acid molecules of the invention, operatively linked to at least one transcription control sequence. In one aspect, the recombinant nucleic acid molecule comprises a plasmid vector. In another aspect, the recombinant nucleic acid molecule comprises a multicistronic vector such that the antigen and mutated E1A protein are expressed as individual proteins. Yet another embodiment of the present invention relates to a composition comprising a mutated E1A protein and a tumor antigen, wherein the mutated E1A protein and tumor antigen are expressed by such a vector.

In yet another aspect, the first nucleic acid sequence is operatively linked to the second nucleic acid sequence to produce a fusion protein comprising the antigen linked to the mutated E1A protein. In this aspect, the first nucleic acid sequence can be operatively linked to the second nucleic acid sequence via a spacer peptide.

In another aspect, the vector is a viral vector, including, but not limited to, a recombinant, non-replicating adenoviral vector. In this aspect, the first nucleic acid sequence encoding an antigen can be cloned into the E3 gene of the adenovirus genome. In one such aspect, the adenovirus is adenovirus serotype 5 and wherein amino acid positions 124-127 of E1A (SEQ ID NO:2) have been deleted.

Another embodiment of the present invention relates to a fusion protein comprising a mutated E1A protein that is covalently linked to an antigen, wherein the fusion protein is expressed by any of the above-referenced recombinant nucleic acid molecules of the present invention. Yet another embodiment of the present invention relates to a composition comprising such a fusion protein of the present invention.

Another embodiment of the present invention relates to a method to produce a vaccine, comprising a first step of (a) culturing a host cell transfected with a recombinant nucleic acid molecule to produce an antigen and a mutated Group C adenoviral E1A protein, the recombinant nucleic acid molecule comprising: (i) a first nucleic acid sequence encoding a vaccinating antigen; and (ii) a second nucleic acid sequence encoding a mutated Group C adenoviral E1A protein, wherein the amino acid sequence of the mutated E1A protein differs from the amino acid sequence of a wild-type E1A protein by at least one mutation, wherein the at least one mutation comprises a mutation in the CR2 region of E1A that abolishes the ability of the mutated E1A protein to bind to pRb, wherein the mutated E1A protein retains an amino acid sequence from the wild-type E1A protein that is sufficient to bind to p300/CBP, and wherein the mutated E1A protein retains an amino acid sequence from the wild-type E1A protein that binds to the C-terminal binding protein (CtBP). The method comprises a second step of (b) recovering the antigen and mutated Group C adenoviral E1A protein from the host cell.

In one aspect of the method, the recombinant nucleic acid molecule comprises a multicistronic vector so that the antigen and mutated E1A protein are expressed as individual proteins. In another aspect of the method, the first nucleic acid sequence encoding the antigen is operatively linked to the second nucleic acid sequence encoding a mutated E1A protein to produce a fusion protein comprising the antigen linked to the mutated E1A protein. In this aspect, the antigen and mutated E1A protein can be linked via an intervening spacer peptide.

Yet another embodiment of the present invention relates to a recombinant modified, non-transforming Group C adenovirus, wherein the genome of the modified adenovirus differs from the genome of a wild-type Group C adenovirus by at least the following modifications: (a) a mutation in the adenoviral genome that renders the recombinant adenovirus non-transforming; (b) a mutation in the genome in the region encoding amino acids of the E1A CR2 domain that is sufficient to abolish binding of the E1A protein to pRb; and (c) an insertion of a nucleic acid sequence encoding an antigen into the E3 region of the genome. The modified adenovirus retains a portion of the E1 genome that encodes an amino acid sequence from the wild-type E1A protein that is sufficient to bind to p300/CBP, and the modified adenovirus retains aportion of the E1 genome that encodes an amino acid sequence from the wild-type E1A protein that binds to the C-terminal binding protein (CtBP). In one aspect, the wild-type adenovirus is selected from the group consisting of adenovirus serotype 2 and adenovirus serotype 5. In another aspect, the portion of the genome encoding amino acids 124-127 of E1A (SEQ ID NO:2 or SEQ ID NO:6) is deleted. In another aspect, the recombinant modified adenovirus has an E1A protein comprising an amino acid sequence that differs from the amino acid sequence of the wild-type E1A by a deletion of amino acids in the CR2 region that abolishes the ability of the mutated E1A protein to bind to pRb. In another aspect, the recombinant modified adenovirus has an E1A protein comprising an amino acid sequence that differs from the amino acid sequence of the wild-type E1A by a substitution or insertion of amino acids in the CR2 region that abolishes the ability of the mutated E1A protein to bind to pRb. In yet another aspect, the recombinant modified adenovirus has an E1A protein comprising an amino acid sequence that is identical to amino acid sequence of the wild-type E1A by retention of amino acid residues in the first 60 amino acid residues of the N-terminus of the protein that are sufficient to bind to p300/CBP.

Yet another embodiment of the present invention relates to a method for eliciting an antigen specific immune response in a patient, comprising administering to the patient any of the above-referenced isolated nucleic acid molecules encoding a mutated E1A protein of the invention, any of the above-identified fusion proteins of the present invention, any of the above-referenced compositions of the present invention, or any of the above-referenced recombinant adenoviruses of the present invention.

Yet another embodiment of the present invention relates to a method for treating a patient that has a tumor, comprising administering to the patient any of the above-referenced isolated nucleic acid molecules of the present invention, any of the above-referenced fusion proteins of the invention, any of the above-referenced compositions of the invention, or any of the above-referenced recombinant adenoviruses of the invention, wherein the antigen is a tumor antigen.

### BRIEF DESCRIPTION OF THE FIGURES

FIG. 1A is a line graph showing NK cell killing of MCA-102 lines transfected with wild type E1A and E1A-ΔRb, but not with E1A-Δp300.

FIG. 1B is a schematic representation of an E1A gene showing the location of E1A-ΔRb, E1A-Δp300 and immunodominant E1A-CTL (D^{b}) epitope in H-2^{b} mice.

FIG. 2A is a bar graph showing that bone marrow derived macrophages kill E1A, but not E7-expressing murine (MCA-102, 3T3) and human cells (H4).

FIG. 2B is a bar graph showing that bone marrow derived macrophages kill E1A-expressing cells predominately through the elaboration of nitric oxide (NO); (L-NAME=N^{G}-Monomethyl-L-Arginine Monoacetate, an inhibitor of nitric oxide synthetase (NOS)).

FIG. 2C is a bar graph showing that E1A-Δp300-expression fails to sensitize MCA-102 cells to killing by activated macrophages.

FIG. 2D is a bar graph showing that E1A-expression sensitizes cells to killing by SNAP, an NO donor, and that NO-dependent killing of E1A-expressing cells is inhibited by incubation with Z-VAD-FMK, a caspase inhibitor.

FIG. 3A is a line graph showing killing of various target cells by cytotoxic T lymphocytes (CTL) generated in C57BL/6 mice by immunization with MCA-102-E1A cells.

FIG. 3B is a line graph showing killing of various target cells by cytotoxic T lymphocytes (CTL) generated in C57BL/6 mice by immunization with MCA-102-E1A-Δp300 cells.

FIG. 3C is a line graph showing killing of various target cells by cytotoxic T lymphocytes (CTL) generated in class II MHC knockout (KO) mice by immunization with MCA-102-E1A-Δp300 cells.

FIG. 4 is a bar graph showing that E1A represses expression of phosphatidylserine receptor on MCA tumor cell lines.

FIG. 5 is a bar graph showing that E1A represses expression of transforming growth factor-β(TGFβ) following stimulation of phosphatidylserine receptor.

### DETAILED DESCRIPTION OF THE INVENTION

The present invention generally relates to the discovery by the present inventor of a molecular basis for differences in viral oncoprotein immunogenicity. Specifically, the inventor has shown that the decreased tumorigenicity in tumor cells expressing E1A from non-oncogenic adenoviruses is dependent on an intact T cell and NK cell antitumor immune response, and has elucidated the structure and functionality in the E1A protein that are required for the immunologically mediated rejection of E1A-expressing cells.

Although human papillomaviruses (HPV) and adenoviruses (Ad) both transform cells by expressing functionally related oncogenes (HPV-E7/E6; Ad-E1A/E1B), only HPV are oncogenic in humans. The inventor hypothesized that in part, the dissimilar oncogenicities of adenoviruses (Ad) and human papillomaviruses (HPV) relate to the vigorous cellular immune response that is triggered in response to cells expressing Ad E1A, which is the primary immortalizing gene of Ad. In comparison to cells expressing E1A, cellular immunity to HPV E7-expressing cells is weak. In support of this hypothesis, the inventor developed an animal model that directly compared the immunogenicities of the adenovirus serotype 5 E1A and human papillomavirus 16 E7 oncoproteins. Studies utilizing this model demonstrated that E7-expressing cells are resistant to rejection by animals with competent NK cell and T cell responses. Conversely, E1A-expressing tumor cells were over 1,000 times less tumorigenic in immunocompetent mice than either E7-expressing cells. The decreased tumorigenicity of E1A-expressing tumor cells was entirely dependent on an intact T cell and NK cell antitumor immune response. These data are summarized in Routes et al., Virology: 277:48-57, 2000. Furthermore, the inventor found that, in contrast to E7, E1A expression in tumor cells: 1) upregulated the expression of Rae-1δ, a ligand for the activating NK cell receptor NKG2D; 2) markedly enhanced the expression of Hsp70; 3) sensitized cells to killing by immune effector cells (NK cells, macrophages) and immune effector mechanisms including Fas, perforin, TNF-α, TRAIL and nitric oxide; and 4) inhibited the anti-apoptotic, NFκB and AKT signaling pathways. Moreover, genetic mapping studies performed by the inventor has shown that the capacity of E1A to bind the cellular proteins, p300/CBP (a property that is lacking in E7) is required for the immunologically-mediated rejection of E1A-expressing cells and for upregulation of Rae-1 and Hsp70, and correlates with sensitization of cells to immune-mediated killing mechanisms.

Therefore, the present inventor has determined that the difference in the immunogenicities of E1A- versus E7-expressing tumor cells is based on a “target and delivery” function of E1A that is not found in E7. First, E1A, and not E7, is able to target cells for destruction by components of the innate immune response due to the upregulation of Rae-1 along with the inhibition of AKT and NFκB, anti-apoptotic, cellular defense pathways. Second, E1A, and not E7, upregulates Hsp70, which results in the efficient delivery of E1A antigenic peptides to antigen presenting cells (APC) following destruction by components of the innate immune response. Third, E1A inhibits tumor cell TGF-β production. TGF-β is a potent, anti-inflammatory cytokine known to inhibit anti-tumor immune responses (Nat Rev Immunol 2(1):46-53, 2002; Cancer Res. 59:1273-77, 1999; Nature Medicine 7:1118-1122, 2001). E1A not only inhibits baseline levels of TGF-β, but also inhibits levels induced following interaction with the phosphatidylserine receptor (PtdSerR), which recognizes apoptotic cells. Interaction of the PSR receptor with apoptotic cells typically induces large amounts of TGF-β and could contribute to potent immunosuppressive effects in the tumor microenvironment (J Clin Invest. 109(1):41-50, 2002; J Cell Biol. 155(4):649-59, 2001; J Clin Invest. 108(7):957-62, 2001; Nature 405(6782):85-90, 2000). As shown in Example 7, this latter activity of E1A is mediated in part by the ability of E1A to downregulate the expression of the PtdSerR.

The present inventor sets forth the following model for this “target and delivery” function that is believed to be responsible for E1A immunogenicity. The E1A oncoprotein is expressed in adenovirus-transformed cells. E1A is a multifunctional nuclear protein that dramatically alters cellular transcription. By binding and altering the coactivator function of p300/CBP, E1A inhibits the AKT and NFκB cellular anti-apoptotic defense pathways and activates transcription of heat shock proteins (in particular Hsp70) and NKG2D ligands (Rae-1 in murine cells). As a result of the inhibition of AKT and NFκB in conjunction with the upregulation of NKG2D ligands, E1A-expressing tumor cells are targeted for destruction by components of the innate immune response (NK cells, macrophages). Following the lysis of E1A-expressing tumor cells, heat shock proteins, and in particular Hsp70, efficiently deliver E1A-antigenic peptides to dendritic cells (DC) by interacting with specific receptors that recognize Hsp70 (CD91, TLR2/4). Ligand binding by CD91 and TLR2 or TLR4 also activates DC. Activation of DC leads to the expression of co-stimulatory molecules and efficient priming of naive, antigen specific T cells. As discussed above, E1A also inhibits tumor cell TGF-β production and TGF-β production following interaction of apoptotic cells with the PtdSerR (see Example 7). This latter activity of E1A interrupts this potent immunosuppressive activity of tumor cells thereby creating an optimal microenvironment for the generation of a robust anti-tumor immune response.

The present inventor's research has revealed that one major difference between the E1A and E7 oncoproteins that influences immunogenicity is the ability of E1A, and not E7, to bind the cellular coadaptor molecules, p300/CBP. The present inventor has shown that tumor cells expressing a form of mutated form of E1A that is unable to bind p300/CBP (E1A-Δp300), like E7, do not inhibit the NFκB/AKT pathways, do not upregulate HSP70 or Rae-1, and are resistant to killing by NK cells and macrophages. Furthermore, tumor cells expressing E1A-Δp300or E7 are far less immunogenic than tumor cells expressing E1A. Differences in the immunogenicities of the E1A and E7 oncoproteins directly effect primary tumor development in a murine model. The present inventor is believed to be the first to appreciate that the ability of E1A to bind to p300/CBP is essential for E1A immunogenicity.

Therefore, it is believed that E1A-expression targets tumor cells for destruction by innate effector mechanisms and efficiently delivers antigens to APCs, by upregulating activating ligands (Rae-1, Hsp70 ) for receptors on expressed on NK cells and macrophages (NKG2D), and by inhibiting the AKT and NFκB pathways, thereby sensitizing cells to killing mechanisms utilized by these immune effector cells (TNFα, TRAIL, perforin, Fas). Finally, E1A induces the production of Hsps, in particular Hsp70, thereby efficiently delivering E1A antigenic peptides to and activating APCs.

As discussed above in detail, other investigators have suggested the use of E1A or particular E1A mutants or fragments to provide a tumor suppressive (anti-oncogenic or anti-tumorigenic) effect. However, although a tumorigenic effect may be observed in the cell that has been transfected with the E1A, there is no evidence that the introduction of E1A into tumor cells leads to the induction of tumor-specific immune responses, which would be more desirable as a therapeutic strategy, since tumor-specific immune responses are directed against both transfected and non-transfected tumor cells. Indeed, the present inventor has shown that although E1A is highly immunogenic, expression of E1A in tumor cells fails to elicit a measurable, protective tumor-specific immune response. The conventional approaches to deliver E1A (or mutants or fragments thereof) into tumor cells (e.g., transfection with the E1A gene, liposomal delivery of E1A protein or infection with adenovirus) are designed to lead to the expression of high levels of E1A protein within tumor cells. This high level of expression is apparently necessary to induce the anti-oncogenic effect of E1A reported by others. However, because E1A is overexpressed in comparison to the endogenous tumor antigens, this conventional approach leads to the development of specific immunity to E1A but not to the endogenous tumor antigen. This effect is most likely due to stoicheometery; the excess of E1A protein within tumor cells leads to efficient presentation of E1A peptides on class I MHC molecule while excluding the presentation of endogenous tumor antigens. Consequently, T cell-specific immune responses are directed against E1A and not against the tumor antigens. The present inventor has illustrated this effect in the experiments described in the Examples.

Therefore, to optimize the therapeutic use of E1A, the present inventor has discovered a construct and a method that can harness the anti-oncogenic activity of E1A (which requires high levels of expression of E1A) and simultaneously enhance, not inhibit, the immunogenicity of endogenous tumor antigens. Specifically, the present inventor has discovered the molecular basis for E1A immunogenicity and has used this knowledge to develop a mutated E1A that is non-transforming, and is immunogenic and anti-oncogenic. The mutated E1A is produced in a construct together with an immunizing antigen such that expression levels of the mutated E1A and antigen are linked and immunogenicity of the antigen is enhanced. This novel construct and the associated compositions and methods have wide therapeutic value for vaccine development and the treatment of cancer and other diseases (e.g., bacterial, viral, fungal, or parasitic infections) in which robust Th1 immune responses are needed, but in which weak Th1 immune responses are normally produced (e.g., HIV infection, hepatitis B infection, hepatitis C infection).

The coupling of the mutated E1A to antigen expression can be achieved by several methods, each of which is described in detail below. First, the mutated E1A and antigen are expressed as a chimeric protein or fusion protein by producing a nucleic acid molecule that encodes both proteins in frame with one another such that the proteins are structurally linked and expressed from the same promoter. An example of such a construct is described in Example 6. Second, the mutated E1A and antigen are expressed by a single construct using a bicistronic or multicistronic vector, wherein the proteins are produced as separate proteins but where the expression of the proteins is coupled so that similar expression time and levels are achieved. Third, the mutated E1A and antigen are expressed by a recombinant adenovirus, where a nucleic acid sequence encoding the antigen is introduced into the genome of an adenovirus that has the appropriate modifications to the gene encoding E1A.

Therefore, one embodiment of the invention relates to an isolated nucleic acid molecule, comprising: (a) a first nucleic acid sequence encoding an antigen; and (b) a second nucleic acid sequence encoding a mutated Group C adenoviral E1A protein. The amino acid sequence of the mutated E1A protein differs from the amino acid sequence of a wild-type E1A protein by at least one mutation in the CR2 region that abolishes the ability of the mutated E1A protein to bind to pRb, retains an amino acid sequence from the wild-type E1A protein that is sufficient to bind to p300/CBP, and retains an amino acid sequence from the wild-type E1A protein that is sufficient to bind to C-terminal binding protein (CtBP). The E1A protein is non-transforming and is immunogenic and as well as retains anti-oncogenic activity.

In accordance with the present invention, an isolated nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to modified (mutated) nucleic acid molecules in which, as compared to the natural or wild-type sequence, nucleotides have been inserted, deleted, substituted, and/or inverted in such a manner that such modifications (mutations) result in a nucleic acid sequence that encodes the desired mutated E1A protein as described herein.

A nucleic acid molecule homologue (e.g., a nucleic acid molecule encoding a mutated E1A protein of the present invention) can be produced using a number of methods known to those skilled in the art (see, for example, Sambrook et al., Molecular Cloning: A Laboratory Manual, Cold Spring Harbor Labs Press, 1989). For example, wild-type nucleic acid molecules can be modified or nucleic acid molecules encoding mutated proteins can be created using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids, for example, by screening for the function of the protein encoded by the nucleic acid and/or by hybridization with a wild-type gene.

The minimum size of a nucleic acid molecule of the present invention is a size sufficient to form a probe or oligonucleotide primer that is capable of forming a stable hybrid (e.g., under moderate, high or very high stringency conditions) with the complementary sequence of a nucleic acid molecule useful in the present invention, or of a size sufficient to encode an amino acid sequence for a mutated E1A protein and/or an antigen according to the present invention (see discussion below). The nucleic acid molecule may also include regulatory regions, linker sequences, vector sequence or other sequence as necessary to provide a nucleic acid molecule according to the present invention. The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a sequence sufficient to encode the proteins of the invention and since the size of the nucleic acid molecule encoding such proteins can be dependent on nucleic acid composition and whether regulatory regions and/or other sequence are included (e.g., linkers, vector sequence, etc.).

In one embodiment, a nucleic acid molecule of the present invention comprises a nucleic acid sequence encoding a mutated E1A protein. According to the present invention, a mutated protein (e.g., a mutated E1A protein), which can also be referred to as a homologue of a wild-type E1A protein, is a protein or polypeptide that has an amino acid sequence which differs from the naturally occurring (wild-type or reference) amino acid sequence (i.e., of the reference protein or domain) in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). According to the present invention, the terms “modification” and “mutation” can be used interchangeably, particularly with regard to the modifications/mutations to the amino acid sequences of E1A (or nucleic acid sequences) described herein. Preferred mutated E1A proteins are described in detail below. For any mutant or homologue protein (e.g., including a homologue or derivative of an antigen, for example) it is noted that homologues can include synthetically produced homologues, naturally occurring allelic variants of a given protein or domain, or homologous sequences from organisms other than the organism from which the reference sequence was derived.

In one embodiment of the present invention, any of the amino acid sequences described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

According to the present invention, the term “contiguous” or “consecutive”, with is regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST:

a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch =−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, is refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

Referring now to the specific embodiments of the invention, an isolated nucleic acid molecule of the present invention includes a first and a second nucleic acid sequence. The first nucleic acid sequence encodes an antigen, and the antigen can be literally any antigen against which the elicitation of an immune response is desired. Particularly preferred antigens for use in the present invention include those antigens against which a relatively weak immune response is typically generated in vivo or in vitro under normal immunization or culture conditions, respectively. The present invention takes advantage of the ability of the mutated El A portion of the construct to induce an immune response against not only itself, but against the protein with which it is expressed. According to the present invention, the terms “immunogen” and “antigen” can be used interchangeably, although the term “antigen” is primarily used to describe a protein (or portion thereof, peptide, glycoprotein or epitope) which elicits a humoral and/or cellular immune response (i.e., is antigenic). The term “immunogen” is primarily used to describe a protein (including portions thereof, peptides, glycoproteins and epitopes) which elicits a humoral and/or cellular immune response in vivo, such that administration of the immunogen to an animal results in an immunogen-specific (antigen-specific) immune response against the same or similar proteins that are encountered within the tissues of the animal. As such, to vaccinate an animal means that an immune response is elicited against the immunogen as a result of administration of the immunogen. Vaccination preferably results in a protective or therapeutic effect, wherein subsequent exposure to the immunogen (or a source of the immunogen) elicits an immune response against the immunogen (or source) that preferably reduces or prevents a disease or condition in the animal. The concept of vaccination is well known in the art. The immune response that is elicited by administration of an immunogen according to the present invention (e.g., via a vaccine, isolated nucleic acid molecule or composition of the present invention) can be any detectable increase in any facet of the immune response (e.g., cellular response, humoral response, cytokine production), as compared to in the absence of the administration of the immunogen.

According to the present invention, the general use herein of the term “immunogen” or “antigen” refers to any portion of a protein (peptide, partial protein, full-length protein), wherein the protein is naturally occurring or synthetically derived, to carbohydrate antigens, to lipid antigens, or to any combination of any of the above-mentioned antigens, wherein the antigen elicits a humoral and/or cellular immune response. An epitope is defined herein as a single antigenic site within a given antigen that is sufficient to elicit an immune response. Those of skill in the art will recognize that T cell epitopes are different in size and composition from B cell epitopes or NK cell epitopes, and that epitopes presented through the Class I MHC pathway differ from epitopes presented through the Class II MHC pathway. An antigen can be as small as a single epitope, or larger, and can include multiple epitopes. As such, the size of an antigen or immunogen can be as small as about 5-12 amino acids (e.g., a peptide) and as large as: a full length protein, including a multimer and fusion proteins, or chimeric proteins. In preferred embodiments, the antigen is selected from the group of a tumor antigen, an allergen or an antigen of an infectious disease pathogen (i.e., a pathogen antigen).

A tumor antigen includes tumor antigens having epitopes that are recognized by T cells, tumor antigens having epitopes that are recognized by B cells, tumor antigens that are recognized by NK cells, tumor antigens that are exclusively expressed by tumor cells, and tumor antigens that are expressed by tumor cells and by non-tumor cells. Tumor antigens can include a tumor antigen including a protein or glycoprotein from a tumor cell or an epitope from a tumor antigen, and portions thereof. Antigens useful in the present invention can include any combination of epitopes, including from the same tumor antigen, from different tumor antigens, from different tumor cells, and from different individuals. Preferably, tumor antigens useful in the present method have at least one T cell, B cell or NK cell epitope.

In a preferred embodiment, a tumor antigen includes a tumor antigen from a cancer including, but not limited to, melanomas, squamous cell carcinoma, breast cancers, cervical cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

According to the present invention, a pathogen antigen that is useful in the present invention includes any antigen from an infectious disease pathogen that can include pathogen antigens having epitopes that are recognized by T cells, pathogen antigens having epitopes that are recognized by B cells, pathogen antigens having epitopes that are recognized by NK cells, pathogen antigens that are exclusively expressed by pathogens, and pathogen antigens that are expressed by pathogens and by other cells. Pathogen antigens can include secreted toxins produced by the pathogen. Preferably, pathogen antigens useful in the present method have at least one T cell, B cell and/or NK cell epitope and are exclusively expressed by pathogens (i.e., and not by the endogenous tissues of the infected mammal). Therefore, delivery of the pathogen antigen to a tissue of an animal elicits an antigen-specific immune response against the pathogen in the tissues of the animal. In some instances, an antigen can be derived from organisms which may not be ordinarily considered to be pathogenic in a mammal, but against which immunization is nonetheless desired.

According to the present invention, a pathogen antigen includes, but is not limited to, an antigen that is expressed by a bacterium, a virus, a parasite, a prion or a fungus. Preferred pathogen antigens for use in the method of the present invention include antigens which cause a chronic or an acute infectious disease in a mammal. For example, some preferred pathogen antigens for use in the present method are antigens from pathogens that cause chronic infections, including, but not limited to, immunodeficiency virus (HIV), Mycobacterium tuberculosis, herpesvirus, papillomavirus, Leishmania, Toxoplasma, Cryptococcus, Blastomyces, Histoplasma, and Candida. Also included are antibiotic resistant strains of bacteria that can cause chronic infections, such as Staphylococcus, Pseudomonas, Streptococcus, Enterococcus, and Salmonella. Additionally, for immunization against acute disease, preferred pathogens from which antigens can be derived include, but are not limited to, Bacillus anthracis, Francisella, Yersenia, Pasteurella, small pox, and other gram negative and gram positive bacterial pathogens.

Examples of viral antigens to be used in a vaccine of the present invention include, but are not limited to, env, gag, rev, tar, tat, nucleocapsid proteins and reverse transcriptase from immunodeficiency viruses (e.g., HIV, HTLV-1, HTLV-2); HBV surface antigen and core antigen; HCV antigens; influenza nucleocapsid proteins; parainfluenza nucleocapsid proteins; human papilloma type 16 E6 and E7 proteins; Epstein-Barr virus LMP-1, LMP-2 and EBNA-2; herpes LAA and glycoprotein D; as well as similar proteins from other viruses.

According to the present invention, an allergen antigen includes a full-length allergen, a portion of the allergen or a homologue of the allergen protein, and includes allergens having epitopes that are recognized by T cells, allergens having epitopes that are recognized by B cells, allergens having epitopes that are recognized by NK cells, and any allergens that are a sensitizing agent in diseases associated with allergic inflammation. Preferred allergens to use in the present invention include plant pollens, drugs, foods, venoms, insect excretions, molds, animal fluids, animal hair and animal dander. Sensitization to an allergen refers to being previously exposed one or more times to an allergen such that an immune response is developed against the allergen. Responses associated with an allergic reaction (e.g., histamine release, rhinitis, edema, vasodilation, bronchial constriction, airway inflammation), typically do not occur when a naive individual is exposed to the allergen for the first time, but once a cellular and humoral immune response is produced against the allergen, the individual is “sensitized” to the allergen. Allergic reactions then occur when the sensitized individual is re-exposed to the same allergen (e.g., an allergen challenge). Once an individual is sensitized to an allergen, the allergic reactions can become worse with each subsequent exposure to the allergen, because each re-exposure not only produces allergic symptoms, but further increases the level of antibody produced against the allergen and the level of T cell response against the allergen.

In another embodiment, an antigen for use in the present invention includes a normal, self-antigen, wherein immunization against such an antigen induces a therapeutic outcome, For example, there might be conditions under which breaking self tolerance, and therefore, eliciting an immune response against a self-antigen (one to which the mammal's immune system is normally tolerant) would be therapeutic. Such self-antigens include, but are not limited to, growth factors, signaling molecules, and normal cells.

Other antigens useful in the present invention and combinations of antigens will be apparent to those of skill in the art. The present invention is not restricted to the use of the antigens described above.

With regard to the second nucleic acid sequence encoding a mutated Group C adenoviral E1A protein, first, a Group C adenoviral E1A protein refers to the E1A protein from any adenovirus that is characterized as belonging to Group C. Group C adenoviruses include at least adenovirus serotype 2 (Ad2) and adenovirus serotype 5 (Ad5) and are non-oncogenic to transfected host cells. The wild-type E1A protein and gene encoding the same have been well documented among the adenoviral serotypes. According to the present invention, the “wild-type” gene or protein refers to the gene or protein, respectively, as it most commonly occurs in nature (i.e., the nucleic acid or amino acid sequence has not been changed by mutation or other intervention). A mutant E1A protein (mutated E1A protein) differs from the wild-type sequence by at least one modification in the sequence (described in detail below).

According to the present invention, an E1A mutated protein (or portion thereof) is anti-oncogenic if the presence of the E1A protein in a cell (e.g., by transfection or infection) does not result in a significant induction of tumor growth by the host cells and/or results in sensitivity of the host cells to killing by activated macrophages, NK cells and/or T cells. The present inventors have shown that the E1A-p300 binding site will sensitize cells to killing by macrophages. However, sensitivity to macrophage killing alone is not sufficient to render E1A anti-oncogenic. The present inventor, without being bound by theory, believes that the anti-oncogenicity of E1A is primarily related to the immunogenicity of E1A (e.g., the ability to induce innate (NK cells and macrophages) and specific (T cells) immunity). Therefore, the p300 binding site, plus additional regions of E1A as described herein, result in sensitivity of host cells to NK and T cell killing and are required for the anti-oncogenic effect of E1A.

According to the present invention, an E1A mutated protein (or portion thereof), or any antigen used in the invention, is immunogenic if the E1A protein or other antigen is capable of detectably inducing (eliciting, stimulating, increasing, upregulating) any facet of the immune response (e.g., cellular response, humoral response, cytokine production), as compared to in the absence of the mutated E1A protein or antigen, respectively, in vivo or in vitro, when the E1A protein is expressed by a host cell, administered to a host cell, or otherwise cultured under appropriate conditions. Appropriate conditions are physiological conditions or culture conditions which provide an environment in which an immune response can occur if immunogenic proteins and the appropriate immune system cells are in contact and which minimizes factors that are inhibitory to immune responses. More specifically, eliciting an immune response generally refers to regulating cell-mediated immunity (e.g., helper T cell (Th) activity, cytotoxic T lymphocyte (CTL) activity, NK cell activity) and/or humoral immunity (e.g., B cell/immunoglobulin activity). As used herein, effector cell immunity generally refers to increasing the number and/or the activity of effector cells in response to the presence of an immunogen in an animal to which an immunogen (e.g., a mutated E1A protein) is administered or in a culture of effector cells with which the immunogen is contacted. In particular, T cell activity refers to increasing the number and/or the activity of T cells in response to the presence of the immunogen. Similarly, NK cell activity refers to increasing the number and/or activity of NK cells. According to the present invention, an effector cell includes a helper T cell, a cytotoxic T cell, a B lymphocyte, a macrophage, a monocyte and/or a natural killer cell. The upregulation (elicitation, induction) of an immune response can be measured using techniques well-known to those in the skill of the art. For example, the ability of a mutated E1A or other immunogen to activate effector cells can be measured by measuring markers of activation on the cells, or by using cytokine assays, proliferation assays, cytotoxicity assays (e.g., measuring destruction of targets), or measuring induction of antibody production (B cells).

As discussed above, the present inventor has shown that E1A (and mutated E1A proteins according to the present invention) is particularly immunogenic because it comprises the p300 binding site. While this site alone is not sufficient for the immunogenicity and anti-oncogenicity of E1A, it is necessary for these characteristics. This site, in conjunction with additional portions of E1A (e.g., CR3 and/or at least a portion of exon 2), is believed, without being bound by theory, to be associated with the ability of E1A to upregulate molecules that target cells expressing E1A for destruction by components of the innate immune response and upregulate molecules (e.g., Hsp70, NKG2D ligands) that result in the efficient delivery of E1A antigenic peptides to antigen presenting cells (APC) so that an effective immune response is elicited against the E1A (and thus the cell expressing E1A). The present inventor has also discovered that E1A expression: 1) inhibits baseline TGF-β production in tumor cells; and 2) inhibits production of TGF-β following ligation of the phosphatidylserine receptor (PSR) on tumor cells. TGF-β is an anti-inflammatory, anti-immunogenic cytokine utilized by tumor cells to inhibit anti-tumor immunity. The ability of E1A to inhibit the baseline and PSR-induced production of TGF-β by tumor cells interrupts the immunosuppressive activity of tumor cells, thereby creating an optimal microenvironment for the generation of a robust anti-tumor immune response. Although it is known that E1A blocks signaling through the TGF-β receptor (not production) (J. Biol Chem 269(41):25392-9, 1994), there are no published observations that E1A can block TGF-β production following ligation of the PSR. Therefore, to the best of the present inventor's knowledge, these are novel findings.

The nucleic acid sequence of the adenovirus serotype 5 genome that contains E1A-encoding sequence is represented as GenBank Accession No. gi58484, incorporated herein by reference in its entirety. Within the sequence described in this accession number, the coding region for the 13S form of E1A (32 kD protein from the 13S RNA) is formed by joining nucleotides 560-1112 (exon 1) and 1229-1545 (exon 2) of the genomic sequence of Accession No. gi58484. The amino acid sequence for Ad5 13S E1A is found in GenBank Accession No. CAB40663.1 and is represented herein by SEQ ID NO:2. SEQ ID NO:2 is encoded by a nucleic acid sequence represented herein by SEQ ID NO: 1 (i.e., SEQ ID NO: 1 represents the joined nucleotides 560-1112 (exon 1) and 1229-1545 (exon 2) of the genomic sequence of Accession No. gi58484). The coding region for the 12S form of E1A (26 kD protein from the 12S RNA) is formed by joining nucleotides 560-974 and 1229-1545 of the genomic sequence of Accession No. gi58484. The amino acid sequence for Ad5 12S E1A is found in GenBank Accession No. CAB40664.1 and is represented herein by SEQ ID NO:4. SEQ ID NO:4 is encoded by a nucleic acid sequence represented herein by SEQ ID NO:3 (i.e., SEQ ID NO:3 represents the joined nucleotides 560-974 and 1229-1545 of the genomic sequence of Accession No. gi58484). The 13S form and the 12S form of Ad5 E1A are similar in that they are identical over the first 132 amino acids and the last 104 amino acids. These forms differ in that the 13S form contains an additional internal region of amino acids known as the conserved region 3 (CR3) (amino acid positions 143-188 of SEQ ID NO:2).

The nucleic acid sequence of the adenovirus serotype 2 genome is represented as GenBank Accession No. gi209811, incorporated herein by reference in its entirety. Within this accession number, the coding region for the 13S form of E1A (32 kD protein from the 13S RNA) is formed by joining nucleotides 559-1111 (exon 1) and 1226-1542 (exon 2) of the genomic sequence of Accession No. gi209811. The amino acid sequence for Ad2 13S E1A is found in GenBank Accession No. AAA92199.1 and is represented herein by SEQ ID NO:6. SEQ ID NO:6 is encoded by a nucleic acid sequence represented herein by SEQ ID NO:5 (i.e., SEQ ID NO:5 represents the joined nucleotides 559-1111 and 1226-1542 of the genomic sequence of Accession No. gi20981 1). The coding region for the 12S form of E1A (26 kD protein from the 12S RNA) is formed by joining nucleotides 559-973 (exon 1) and 1226-1542 (exon 2) of the genomic sequence of Accession No. gi209811. The amino acid sequence for Ad2 12S E1A is found in GenBank Accession No. AAA92197.1 and is represented herein by SEQ ID NO:8. SEQ ID NO:8 is encoded by a nucleic acid sequence represented herein by SEQ ID NO:7 (i.e., SEQ ID NO:7 represents the joined nucleotides 559-973 and 1226-1542 of the genomic sequence of Accession No. gi209811). The 13S form and the 12S form are similar in that they are identical over the first 132 amino acids and the last 104 amino acids. These forms differ in that the 13S form contains an additional region of amino acids known as the conserved region 3 (CR3) (amino acid positions 143-188 of SEQ ID NO:6).

Comparison of the amino acid sequences for the adenovirus serotypes 2 and 5 E1A proteins, including both the 13S and the 12S forms, shows the significant homology between the serotypes. The following discussion will further identify the portions of the E1A proteins that are most important for the present invention. Such discussion is intended to provide guidance as to what portions of a wild-type sequence can be modified while maintaining the structural and functional requirements of a mutated E1A protein of the present invention.

The mutated E1A protein encoded by the nucleic acid molecule of the present invention (i.e., encoded by the second nucleic acid sequence) has at least three specific structural elements that are responsible for functional characteristics of the mutated E1A protein according to the present invention. First, the amino acid sequence of the mutated E1A differs from the amino acid sequence of a wild-type E1A protein by at least one mutation of an amino acid residue (e.g., deletion, substitution, addition, derivatization), with the mutation(s) necessarily including at least one mutation in the CR2 region of the protein, wherein the mutation in the CR2 region abolishes the ability of the mutated E1A protein to bind to the product of the retinoblastoma gene, pRb. It is known in the art that through two highly homologous, conserved regions (i.e., conserved region (CR1) and CR2), E1A binds to and inhibits the function of common cell-growth regulatory proteins (e.g. p105 or retinoblastoma gene product, pRb; p107, p130, and cyclin A). In the present invention, in order to render the viral protein non-transforming, a sufficient number of the amino acids in the CR2 region are mutated (e.g., by deletion, substitution, or insertion of other amino acids) such that the ability of the E1A protein to bind to pRb is abolished (e.g., eliminated, prevented, suppressed). According to the present invention, to abolish binding to pRb refers to a substantial abolition (reduction, elimination, prevention, suppression) of binding between pRb and E1A, where the binding affinity of the mutated E 1 A protein for the pRb, as compared to the wild-type E1A protein, is reduced by at least about 50%, and more preferably by at least about 60%, and more preferably by at least about 70%, and more preferably by at least about 80%, and more preferably at least about 90%, and more preferably at least about 95%, and more preferably at least about 96%, and more preferably at least about 97%, and more preferably at least about 98%, and more preferably at least about 99%, and most preferably, wherein binding of the mutated E1A protein to pRb is undetectable using standard binding assays.

The amino acids in the CR2 region of E1A that are important for the binding of E1A to pRb are conserved among the 13S RNA and 12S RNA forms of E1A in both adenovirus serotype 2 and adenovirus serotype 5, and are located at positions 124-127 of the amino acid sequences (SEQ ID NOs:2, 4, 6 or 8). Therefore, in one embodiment, the portion of the mutated E1A that is mutated in order to abolish pRb binding, as compared to the wild-type sequence, includes at least the residues that occur at positions 124-127 in any of the adenovirus E1A sequence (e.g., SEQ ID NOs:2, 4, 6 or 8). Additional portions of the CR2 region can be mutated without impacting the anti-oncogenic or immunogenic properties of the E1A protein. For example, amino acid residues that occur at positions 123-128 can be mutated, or any additional residues within the CR2 region that include at least 124-127 (e.g., 122-127, 120-127, 124-128, etc.), up to the entire CR2 region, can be mutated. The CR2 region of the 13S form of E1A in adenovirus serotype 2 or 5 spans from about amino acid position 120 to about amino acid position 140. The CR2 region of the 12S form of E1A in adenovirus serotype 2 or 5 spans from about amino acid position 120 to about position 132.

According to the present invention, one of skill in the art can determine whether a mutated E1A protein binds to pRb (or indeed binding between any two proteins) or is non-transforming using any binding assays known in the art. For example, a BIAcore machine can be used to determine the binding constant of a complex between an E1A mutated protein and pRb. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of mutated E1A to pRb include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR). In addition, one can determine whether a mutated E1A protein is capable of transforming a host cell (e.g., where the host cell demonstrates transformation characteristics such as formation of foci, anchorage independence, loss of growth factor or serum requirements, change in cell morphology, ability to form tumors when injected into suitable animal hosts, and/or immortalization of the cell). Non-transforming mutated E1A proteins are not capable of causing transformation of a host cell.

Second, the amino acid sequence of the mutated E1A protein must have (i.e., retain, possess, include) an amino acid sequence from the wild-type E1A protein that is sufficient to bind to p300/CBP (i.e., enough of the native sequence and structure is present that p300/CBP will bind to the mutated E1A protein). The present inventor, without being bound by theory, believes that the binding of E1A to p300/CBP is essential for E1A immunogenicity, and that this immunogenicity is directly related to the anti-oncogenicity of E1A. Specifically, the inventor believes that binding of E1A to p300/CBP is related to the ability of E1A to upregulate activating ligands (Rae-1, Hsp70 ) for receptors that are expressed on NK cells and macrophages (NKG2D) and that enable efficient delivery of E1A antigenic peptides to APCs, and to inhibit the AKT and NFκB pathways, thereby sensitizing cells transfected with E1A to killing mechanisms utilized by these immune effector cells (TNFα, TRAIL, perforin, Fas). Therefore, a mutated E1A protein is identical to the wild-type E1A by retaining the portions of the amino acid sequence, in suitable positions with respect to one another, that enable the E1A protein to bind to the p300/CBP complex.

There are at least two regions of the wild-type E1A protein that are required for E1A binding to p300/CBP. The first region is in the N-terminal region of the protein and, while other N-terminal residues are included, this region specifically includes a residue that is highly conserved among adenovirus E1A proteins, the arginine at position 2. Additional amino acids in the N-terminus are also preferably included. Therefore, the mutated E1A protein, at a minimum, includes an arginine at about position 2 of the N-terminal sequence, or at least an arginine within the first 10 amino acids of the sequence. This first region can be extended to maintain at least residues 1-5 of the wild-type protein, or at least 1-10, or at least 1-20, or at least 1-30, or at least 1-40, or at least 1-47, or any number of positions within the first 47 amino acid residues that include the arginine at about position 2, of the wild-type protein residues at those positions.

The second region of the p300/CBP binding site is located from about positions 48-60 of the wild-type sequences for any of the adenovirus serotypes 2 or 5, including both the 12S and the 13S forms. These amino acids are in the CR1 region of E1A, which spans from about amino acid 10 to about 60 in both the 13S and 12S forms of adenovirus serotypes 2 and 5. Therefore, the mutated E1A protein of the invention, includes, at a minimum, the amino acid residues that occur at from about position 48 to about position 60 of the wild-type protein, spatially arranged with respect to the other amino acids in the N-terminal region required for p300/CBP binding (discussed above) so that binding to p300/CBP by the mutated E1A protein is retained.

Therefore, the mutated E1A protein of the present invention includes a sufficient portion of the first and second binding sites for p300/CBP, which are spatially arranged in the mutated E1A sequence in a manner that is similar enough to the spatial arrangement in the wild-type E1A, so that binding to p300/CBP can be achieved. At a minimum, this includes the amino acid residues that occur in positions 2 and 48-60 in the wild-type protein (e.g., with approximately 46 amino acids intervening these residues), and may include additional amino acid residues from the wild-type sequence as discussed above. It is to be understood that the positions of these conserved residues within the mutated E1A protein can vary somewhat from the positions in the wild-type protein, as long as the mutated E1A protein maintains the ability to bind to the p300/CBP protein. For example, one could construct a mutated E1A protein where the arginine that occurs at position 2 of the wild-type protein appears at position 3, 4, 5 or even higher in the mutant. As another example, the second region that spans positions 48-60 of the wild-type protein may, in the mutated protein, be located at positions 44-56 or 50-62. To the extent that intervening residues are required to maintain the approximate distance between the first and second binding regions for p300/CBP (e.g., so that the mutated E1A can bind to p300/CBP), one can construct the mutated E1A accordingly, although it may be possible to modify the identity of the intervening residues, or even those which bind to p300/CBP, at a minimum, by using conservative amino acid substitutions. As another example, one could create a modified sequence for amino acids 1-60 of E1A, wherein at least residue 2 and 48-60, and any other residues necessary to maintain p300 binding are retained (binding determined as described previously herein), and wherein the remaining residues are either also the same as the wild-type E1A residues or a conservative substitution for the wild-type residue. Some residues in the region spanning positions 1-60 may also be deleted, as long as p300 binding is retained.

Conservative substitutions typically include substitutions within the following groups: glycine and alanine; valine, isoleucine and leucine; aspartic acid, glutamic acid, asparagine, and glutamine; serine and threonine; lysine and arginine; and phenylalanine and tyrosine. Substitutions may also be made on the basis of conserved hydrophobicity or hydrophilicity (Kyte and Doolittle, J. Mol. Biol. (1982)157:105-132), or on the basis of the ability to assume similar polypeptide secondary structure (Chou and Fasman, Adv. Enzymol. (1978) 47: 45-148, 1978).

Third, the amino acid sequence of the mutated E1A protein must have (i.e., retain, possess, include) an amino acid sequence from the wild-type E1A protein that is sufficient to bind to the C-terminal binding protein (CtBP) (i.e., enough of the native sequence and structure is present that CtBP will bind to the mutated E1A protein). The C-terminal binding protein is a transcriptional corepressor that has previously been shown to interact with the C-terminal portion of E1A and is required for efficient activation of E1A-responsive genes. It is believed that the interaction between E1A and CtBP may block CtBP-mediated repression in the host cell. The CtBP interacting domain (CID) is located in the second exon of E1A and includes at least a conserved amino acid motif in the extreme C-terminus denoted “PXDLS” (represented by positions 279-283 of SEQ ID NOs:2 and 6; positions 233-237 of SEQ ID NOs:4 and 8). It is believed that more than just this conserved region is required for CtBP binding to E1A, and for the purposes of this invention, the portion of E1A represented by amino acid positions 225-238 of SEQ ID NOs:4 and 8, or by amino acid positions 271-284 of SEQ ID NOs:2 and 6 are considered to be important for CtBP binding (see, e.g., Schaeper et al., (1995), Proc. Natl. Acad. Sci. USA 92(23):10467-10471). In another embodiment, the entire final 44 amino acids of the C-terminus are retained for CtBP binding (represented by amino acids 200-243 of SEQ ID NOs:4 and 8 and amino acids 246-289 of SEQ ID NOs:2 and 6). CtBP binding to E1A can be determined using standard binding assays as described previously herein. In addition, CtBP binding with relation to E1A is described in, e.g., Schaeper et al., 1995, ibid.; Molloy et al., (1998), J. Biol. Chem. 273(33):20867-20876; Molloy et al., (2001), Biochim Biophys Acta 1546(1):55-70; Sollerbrant et al., (1996), Nucl. Acids. Res. 24(13):2578-2584; Sundqvist et al., (2001), Exp Cell Res. 268(2):284-293; and Kladney et al., (2002), Virology 301(2):236-246.

In one embodiment of the invention, the mutated E1A protein retains from the wild-type sequence the amino acids of the CR3 region of the 13S forms of E1A (represented by amino acids 143-188 of SEQ ID NOs:2 and 6). The CR3 region, along with the E1A-p300 binding site, is required for optimal upregulation of Hsp70 by E1A. Hsp70 is a chaperone protein, the presence of which results in the efficient delivery of peptides (e.g. in the invention, of E1A antigenic peptides and antigen peptides, to antigen presenting cells (APC)). This allows for increased immunogenicity (and therefore anti-oncogenicity) of the mutated E1A and constructs comprising the mutated E1A according to the invention, since T-cell mediated immune responses require APC presentation of antigenic peptides. Therefore, although the CR3 region is not absolutely required to be present in a mutated E1A of the invention, it is a preferred embodiment to include this region. In one embodiment, only the exact amino acids from the CR3 region that are required to obtain upregulation of Hsp70 are retained. This can be determined by one of skill in the art by simple mutational analysis, for example.

In further embodiments of the present invention, the mutated E1A can have other modifications which distinguish the mutated protein from the wild-type E1A protein. For example, in one aspect of the invention, a few of the amino acids at the C-terminal end of the protein can be modified or deleted without affecting the anti-oncogenic or the immunogenic properties of the E1A protein. In one aspect, at least the final 1-5 amino acids of the C-terminus (positions 285-289 of SEQ ID NOs:2 and 6 or positions 239-243 of SEQ ID NOs:4 and 8) can be modified (e.g., by deletion, substitution, insertion or derivatization), while maintaining the oncogenicity and immunogenicity of the mutated E1A protein. In another embodiment, any one or more of the amino acid residues can be modified that occur between: (1) the C-terminal end of the CR2 region of either the 12S or the 13S form (about residue 140 of SEQ ID NOs:2 and 6 and about residue 132 of SEQ ID NOs:4 and 8), and either of: (2a) the N-terminal end of the last 44 amino acids of the protein (about residue 246 of SEQ ID NOs:2 and 6 and about residue 200 of SEQ ID NOs:4 and 8), or (2b) the N-terminal end of the important CID residues (about residue 271 of SEQ ID NOs:2 and 6 or about residue 225 of SEQ ID NOs:4 and 8). In yet another embodiment, for the 13S forms, any one or more of the amino acid residues can be modified that occur between: (1) the C-terminal end of the CR3 region (about residue 188 of SEQ ID NOs:2 and 6), and either of: (2a) the N-terminal end of the last 44 amino acids of the protein (about residue 246 of SEQ ID NOs:2 and 6), or (2b) the N-terminal end of the important CID residues (about residue 271 of SEQ ID NOs:2 and 6).

In the embodiments described above, the mutated E1A protein further differs from the wild-type protein by deletion or other modification of at least one amino acid residue within the recited areas, and including any number of additional deletions or modifications (e.g., 2, 3, 4, 5, 10, 15, 20, 28, 32, etc.) in the recited areas. In any of these embodiments, both the anti-oncogenicity and the immunogenicity of the protein is maintained, which can be determined using the assays as described above or as exemplified in the Examples section.

In further embodiments of the invention, as long as the regions and functions of the E1A protein as specified above are modified or retained, other amino acid residues that are not in these regions or involved in the immunogenicity and oncogenicity of the protein may be modified by deletion, substitution or insertion (or even derivatization). Therefore, in one embodiment of the invention, the mutated E1A protein further differs from the wild-type E1A protein by deletion or other modification of at least one amino acid residue from the sequence of E1A that does not participate in p300/CBP binding, in CtBP binding, or optionally, in upregulation of Hsp70 (all described above). In further embodiments, any number of additional deletions or modifications (e.g., 2, 3, 4, 5, 10, 15, 20, 45, 53, etc.) in such less important regions of the sequence can be modified, as long as the immunogenicity and anti-oncogenicity of the mutated E1A protein is retained, which can be determined using the assays known in the art and as described herein.

Some preferred mutated E1A proteins of the invention are described below, although the invention is not limited to these proteins, as can be ascertained from the discussion above. In one embodiment, the mutated E1A comprises, in consecutive order, amino acid positions 1 to about 140 and amino acid positions from about 271 to about 284 of SEQ ID NO:2 or SEQ ID NO:6, wherein amino acids in the CR2 region have been mutated to abolish the ability of the mutated E1A to bind to pRb. In another embodiment, the mutated E1A comprises, in consecutive order, amino acid positions 1 to about 188 and amino acid positions from about 271 to about 284 of SEQ ID NO:2 or SEQ ID NO:6, wherein amino acids in the CR2 region have been mutated to abolish the ability of the mutated E1A to bind to pRb. In yet another embodiment, the mutated E1A comprises, in consecutive order, amino acids 1 to about 123, amino acids from about 128 to about 140, and amino acids from about 271 to about 284, of SEQ ID NO:2 or SEQ ID NO:6. In another embodiment, the mutated E1A comprises, in consecutive order, amino acids 1 to about 123, amino acids from about 128 to about 188, and amino acids from about 271 to about 284, of SEQ ID NO:2 or SEQ ID NO:6. In another embodiment, the mutated E1A comprises, in consecutive order, amino acid positions 1 to about 140 and amino acid positions from about 246 to about 289 of SEQ ID NO:2 or SEQ ID NO:6, wherein amino acids in the CR2 region have been mutated to abolish the ability of the mutated E1A to bind to pRb. In yet another embodiment, the mutated E1A comprises, in consecutive order, amino acid positions 1 to about 188 and amino acid positions from about 246 to about 289 of SEQ ID NO:2 or SEQ ID NO:6, wherein amino acids in the CR2 region have been mutated to abolish the ability of the mutated E1A to bind to pRb. In another embodiment, the mutated E1A comprises, in consecutive order, amino acids 1 to about 123, amino acids from about 128 to about 140, and amino acids from about 246 to about 289, of SEQ ID NO:2 or SEQ ID NO:6. In another embodiment, the mutated E1A comprises, in consecutive order, amino acids 1 to about 123, amino acids from about 128 to about 188, and amino acids from about 246 to about 289, of SEQ ID NO:2 or SEQ ID NO:6. In yet another embodiment, the mutated E1A consists of, in consecutive order, amino acids 1 to about 123, amino acids from about 128 to about 188, and amino acids from about 246 to about 289, of SEQ ID NO:2 or SEQ ID NO:6. In another embodiment, the mutated E1A comprises, in consecutive order, amino acids 1 to about 132, and amino acids from about 225-238 of SEQ ID NO:4 or SEQ ID NO:8, wherein amino acids in the CR2 region have been mutated to abolish the ability of the mutated E1A to bind to pRb. In yet another embodiment, the mutated E1A comprises, in consecutive order, amino acids 1 to about 132, and amino acids from about 200-243 of SEQ ID NO:4 or SEQ ID NO:8, wherein amino acids in the CR2 region have been mutated to abolish the ability of the mutated E1A to bind to pRb. In another embodiment, the mutated E1A comprises, in consecutive order, amino acids 1 to about 123, amino acids from about 128 to about 132, and amino acids from about225-238 of SEQ ID NO:4 or SEQ ID NO:8. In another embodiment, the mutated E1A comprises, in consecutive order, amino acids 1 to about 123, amino acids from about 128 to about 132, and amino acids from about 200-243 of SEQ ID NO:4 or SEQ ID NO:8. In another embodiment, the mutated E1A consists of in consecutive order, amino acids 1 to about 123, amino acids from about 128 to about 132, and amino acids from about 200-243 of SEQ ID NO:4 or SEQ ID NO:8.

In one embodiment of the invention, a mutated E1A protein differs from the wild-type E1A sequence by having an overall amino acid identity to the wild-type sequence of at least about 25%, and in another embodiment at least about 30%, and in another embodiment at least about 35%, and in another embodiment at least about 40%, and in another embodiment at least about 45%, and in another embodiment at least about 50%, and in another embodiment at least about 55%, and in another embodiment at-least about 60%, and in another embodiment at least about 65%, and in another embodiment at least about 70%, and in another embodiment at least about 75%, and in another embodiment at least about 80%, and in another embodiment at least about 85%, and in another embodiment at least about 90%, and in another embodiment at least about 95%, and in another embodiment at least about 96%, and in another embodiment at least about 97%, and in another embodiment at least about 98%, and in another embodiment at least about 99%, wherein the mutated E1A has the minimum characteristics of a mutated E1A protein as described above (i.e., lack of pRb binding, retention of p300/CBP binding, and retention of CtBP binding, and optionally, retention of amino acids of the CR3 region that are sufficient to increase the ability of E1A to upregulate Hsp70 ). Methods to determine percent identity have been discussed above.

In another embodiment of the invention, a mutated E1A protein of the present invention, in addition to having the minimum characteristics of a mutated E1A protein as described above, has an amino acid sequence that is sufficiently similar to a wild-type E1A protein that a nucleic acid sequence encoding the amino acid sequence for the mutated E1A protein is capable of hybridizing under moderate, high, or very high stringency conditions (described below) to (i.e., with) a nucleic acid molecule encoding the wild-type E1A protein (i.e., to the complement of the nucleic acid strand encoding the wild-type E1A protein). Stringent hybridization conditions have been discussed above.

The first nucleic acid sequence encoding an antigen and the second nucleic acid sequence encoding a mutated E1A protein are included in the same nucleic acid molecule construct so that the two protein sequences will be expressed at approximately the same level. As discussed above, the present inventor has discovered that by linking expression of an antigen to expression of E1A, the highly immunogenic properties of E1A can be used to enhance the immunogenicity of the antigen, thereby resulting in an effective immune response against the antigen. The first and second nucleic acid sequences can be provided in the nucleic acid molecule in a variety of ways. First, the two sequences can be operatively linked so that the mutated E1A and antigen are expressed as a chimeric protein or fusion protein. This is achieved by producing a nucleic acid molecule that encodes both proteins in frame with one another such that the proteins are structurally linked and expressed from the same promoter. Second, the two sequences can be included in a single construct using a bicistronic or even a multicistronic vector (e.g., multicistronic vectors can be used to express more than one antigen, for example), wherein the encoded proteins are produced as separate proteins, but where the expression of the proteins is coupled so that similar expression time and levels are achieved. Third, the two sequences can be included in a recombinant adenovirus, where a nucleic acid sequence encoding the antigen is introduced into the genome of an adenovirus that has the appropriate modifications to the gene encoding E1A (i.e., abolition of pRb binding, retention of p300/CBP binding, retention of CtBP binding, and optionally, retention of the requisite portion of CR3 to upregulate Hsp70 ). Methods for producing fusion proteins, bicistronic vectors, and recombinant adenoviruses are known in the art, and some discussion of such methods is provided below.

One embodiment of the present invention relates to a recombinant nucleic acid molecule which comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the antigen and mutated E1A protein) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants. In microbial systems, the term “transformation” is used to describe an inherited change due to the acquisition of exogenous nucleic acids by the microorganism and is essentially synonymous with the term “transfection.” However, in animal cells, transformation has acquired a second meaning which can refer to changes in the growth properties of cells in culture (described above) after they become cancerous, for example. Therefore, to avoid confusion, the term “transfection” is preferably used with regard to the introduction of exogenous nucleic acids into animal cells, and is used herein to generally encompass transfection of animal cells and transformation of plant cells and microbial cells, to the extent that the terms pertain to the introduction of exogenous nucleic acids into a cell. Therefore, transfection techniques include, but are not limited to, transformation, particle bombardment, electroporation, microinjection, lipofection, adsorption, infection and protoplast fusion.

One or more recombinant molecules of the present invention can be used to produce an encoded product (e.g., an antigen and mutated E1A protein, including a fusion or chimeric protein) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cell that can be transfected. Host cells can be either untransfected cells or cells that are already transfected with at least one other recombinant nucleic acid molecule.

In one embodiment, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole (e.g., for ex vivo administration), either of which can be used in a composition, such as a vaccine. A preferred cell to culture is any suitable host cell as described above. Effective culture conditions include, but are not limited to, effective media, bioreactor, temperature, pH and oxygen conditions that permit protein production and/or recombination. An effective medium refers to any medium in which a given host cell is typically cultured. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. Proteins produced according to the present invention are preferably retrieved in “substantially pure” form. As used herein, “substantially pure” refers to a purity that allows for the effective use of the protein as a commercial product or in a vaccine or other therapeutic composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts.

In one embodiment of the invention, the mutated E1A protein and the antigen are expressed as individual proteins. However, in order to effectively produce compositions (e.g., vaccines) that are useful for eliciting an immune response against the antigen for therapeutic benefit, the proteins should be expressed at a substantially similar level and with substantially the same timing. This is most typically achieved by producing a bicistronic vector or even a multicistronic vector (e.g., when more than one antigen is to be expressed), wherein internal ribosome entry sites (IRES), which are cis-acting elements that recruit the small ribosomal submits to an interior initiator codon in the mRNA, are used in the recombinant vector to allow for coupled coexpression of the mutated E1A protein and the antigen. Using such a vector, the first nucleic acid sequence is cloned into one cassette of the vector and the second nucleic acid sequence is cloned into a second cassette of the vector. The vector can then be used as a DNA vaccine (e.g., administration of the DNA to a cell or animal) or to produce a composition of mutated E1A protein and antigen in vitro (e.g., to prepare a protein vaccine). In this embodiment, it is noted that an isolated nucleic acid molecule of the present invention can comprise more than one nucleic acid sequence encoding an antigen, such that one construct can express two or more antigens, if desired, in addition to the mutated E1A protein. Therefore, the expression of multiple antigens (e.g., encoded by second, third, fourth nucleic acid sequences, and so on) can be coupled to the expression of the mutated E1A protein in order to increase the efficacy of a given composition or vaccine comprising the expression products.

In another embodiment of the invention, the mutated E1A protein and antigen are produced as a fusion protein, also referred to herein as a chimeric protein. In this embodiment, the nucleic acid sequence encoding an antigen is in frame with and covalently attached to the nucleic acid sequence encoding the mutated E1A protein so that the expressed protein is a chimera or fusion between the mutated E1A and the antigen. In one aspect, the protein is joined directly to the amino or carboxyl termini of the other protein. In another aspect, the two nucleic acid sequences can be attached to each other by a nucleic acid linker sequence encoding from one to multiple amino acid residues (e.g. referred to herein as a linker peptide or a spacer peptide) which serve to link the expressed proteins together. Such spacer peptides or linkers can enhance the ability of the protein to fold or, in some embodiments, can include a cleavage sequence to enable the proteins to be separated after production, if desired. Suitable linker sequences are known in the art and should not significantly reduce the ability of either the mutated E1A protein or the antigen to function as described for the present invention.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses, but is preferably a recombinant, non-replicating adenoviral vector and most preferably, an adenoviral vector derived from a serotype 2 or serotype 5 adenovirus.

In one embodiment, a recombinant adenovirus is provided, wherein the first nucleic acid sequence encoding an antigen is cloned into the E3 gene of the adenovirus genome. In addition, the E1A gene of the adenovirus genome is mutated to meet at least the minimum requirements of a mutated E1A according to the present invention, and includes the specified regions that are to be retained from the wild-type sequence. Mutated adenoviral vectors can be produced using standard molecular techniques for producing modified nucleic acid molecules as discussed above. Adenoviral vectors are well known in the art and can be readily obtained as starting materials from which to create a recombinant adenoviral vector of the present invention.

The isolated nucleic acid molecule of the present invention, as well as the proteins produced by such molecule are all useful in various compositions of the invention. For example, in one embodiment, the isolated nucleic acid molecule (preferably as part of a recombinant nucleic acid molecule) is useful as a genetic vaccine, wherein administration of the nucleic acid molecule to an animal results in transfection of host cells of the animal with the molecule and expression of the protein(s) expressed by the molecule. In another embodiment, the isolated nucleic acid molecule is used to produce the encoded protein(s) in vitro, which can then be used in a protein vaccine or other therapeutic composition. In yet another embodiment, the isolated nucleic acid molecule can be used to transfect cells ex vivo and then the cells are returned to the patient from which they were removed so that an immune response against the antigen can be elicited in vivo.

In another embodiment of the present invention, a vaccine or therapeutic composition further comprises a pharmaceutically acceptable carrier, which includes pharmaceutically acceptable excipients and/or delivery vehicles, for delivering the recombinant nucleic acid molecule or the proteins (e. g., mutated E1A and antigen produced by an isolated nucleic acid molecule of the present invention) to a patient. As used herein, a pharmaceutically acceptable carrier refers to any substance or vehicle suitable for delivering a therapeutic composition (e.g., a vaccine) useful in a therapeutic method of the present invention (described below) to a suitable in vivo or ex vivo site. Preferred pharmaceutically acceptable carriers are capable of maintaining a recombinant nucleic acid molecule of the present invention in a form that, upon arrival of the nucleic acid molecule to a target cell or tissue, the nucleic acid molecule is capable of entering the cell and being expressed by the cell, whereby the expressed mutated E1A protein and antigen can elicit an immune response against both the E1A protein and the antigen at and/or near the site of the cell or tissue. When the composition is the protein composition (i.e., mutated E1A protein and antigen), preferred pharmaceutically acceptable carriers are capable of maintaining the protein composition in a form that, upon arrival of the protein composition to a target cell or tissue, the proteins are capable of eliciting an immune response at or near the site of the cell and/or tissue. A pharmaceutically acceptable carrier can include a pharmaceutically acceptable excipient. Suitable excipients of the present invention include excipients or formularies useful in a vaccine or other therapeutic composition. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable pharmaceutically acceptable carriers for nucleic acids include, but are not limited to liposomes or other lipid-containing vehicles, viral vectors, ribozymes, gold Is particles, poly-L-lysine/DNA-molecular conjugates, and artificial chromosomes. Natural lipid-containing delivery vehicles include cells and cellular membranes. Artificial lipid-containing delivery vehicles include liposomes and micelles. A delivery vehicle can be modified to target to a particular site in a patient, thereby targeting and making use of a nucleic acid molecule at that site. Suitable modifications include manipulating the chemical formula of the lipid portion of the delivery vehicle and/or introducing into the vehicle a targeting agent (e.g., an antibody or peptide) capable of specifically targeting a delivery vehicle to a preferred site, for example, a preferred cell type.

A liposome delivery vehicle comprises a lipid composition that is capable of delivering a recombinant nucleic acid molecule of the present invention, including both plasmids and viral vectors, to a suitable cell and/or tissue in a patient. A liposome delivery vehicle comprises a lipid composition that is capable of fusing with the plasma membrane of the target cell to deliver the recombinant nucleic acid molecule into a cell. As discussed above, liposome delivery vehicles can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands). Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety).

Suitable pharmaceutically acceptable carriers for protein compositions include, but are not limited to liquid injectables or solids which can be taken up in a suitable liquid as a suspension or solution for injection, liquids that can be aerosolized, capsules, or tablets. In a non-liquid formulation, the excipient can comprise, for example, dextrose, human serum albumin, and/or preservatives to which sterile water or saline can be added prior to administration.

One type of pharmaceutically acceptable carrier includes a controlled release formulation that is capable of slowly releasing a composition of the present invention into an animal. As used herein, a controlled release formulation comprises recombinant nucleic acid molecule or protein composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems.

Also included in the present invention are methods of eliciting an antigen-specific immune response in a patient for the purposes of vaccinating the patient or providing another therapeutic benefit to the patient, such as treating an ongoing disease or condition. This method includes administering to the patient: (1) an isolated nucleic acid molecule according to the present invention (preferably as a recombinant nucleic acid molecule, including a recombinant virus, as described herein); (2) a vaccine or composition containing the mutated E1A protein and antigen produced by the isolated nucleic acid molecule according to the present invention (including fusion or chimeric proteins and proteins expressed by a bicistronic vector); or (3) a recombinant cell transfected with the isolated nucleic acid molecule of the present invention. Such a method can be useful for treating a patient that has or is at risk of developing a tumor, for treating a patient that has or is at risk of developing an infectious disease, for treating a patient that has or is at risk of developing allergic inflammation, or for treating a patient with any disease or condition for which eliciting an immune response against a given antigen may be beneficial.

Accordingly, the method of the present invention preferably elicits an immune response in a patient such that the patient is protected from a disease that is amenable to elicitation of an immune response, including cancer, allergic inflammation and/or an infectious disease. As used herein, the phrase “protected from a disease” refers to reducing the symptoms of the disease; reducing the occurrence of the disease, and/or reducing the severity of the disease. Protecting a patient can refer to the ability of a therapeutic composition of the present invention, when administered to a patient, to prevent a disease from occurring and/or to cure or to alleviate disease symptoms, signs or causes. As such, to protect a patient from a disease includes both preventing disease occurrence (prophylactic treatment, such as by vaccination/immunization) and treating a patient that has a disease or that is experiencing initial symptoms or later stage symptoms of a disease (therapeutic treatment). In particular, protecting a patient from a disease is accomplished by eliciting an immune response in the patient by inducing a beneficial or protective immune response which may, in some instances, additionally suppress (e.g., reduce, inhibit or block) an overactive or harmful immune response. The term, “disease” refers to any deviation from the normal health of a patient and includes a state when disease symptoms are present, as well as conditions in which a deviation (e.g., infection, gene mutation, genetic defect, etc.) has occurred, but symptoms are not yet manifested (e.g., a precancerous condition).

More specifically, a vaccine or therapeutic composition as described herein, when administered to a patient by the method of the present invention, preferably produces a result which can include alleviation of the disease (e.g., reduction of at least one symptom or clinical manifestation of the disease), elimination of the disease, reduction of a tumor or lesion associated with the disease, elimination of a tumor or lesion associated with the disease, prevention or alleviation of a secondary disease resulting from the occurrence of a primary disease (e.g., metastatic cancer resulting from a primary cancer), prevention of the disease, and stimulation of effector cell immunity and/or humoral immunity against the disease.

According to the present invention, an effective administration protocol (i.e., administering a vaccine or therapeutic composition in an effective manner) comprises suitable dose parameters and modes of administration that result in elicitation of an immune response in a patient that has a disease or condition or that may develop a disease or condition, preferably so that the patient is protected from the disease (e.g., by disease prevention or by alleviating one or more symptoms of ongoing disease). Effective dose parameters can be determined using methods standard in the art for a particular disease. Such methods include, for example, determination of survival rates, side effects (i.e., toxicity) and progression or regression of disease. In particular, the effectiveness of dose parameters of a therapeutic composition of the present invention when treating cancer can be determined by assessing response rates. Such response rates refer to the percentage of treated patients in a population of patients that respond with either partial or complete remission. Remission can be determined by, for example, measuring tumor size or microscopic examination for the presence of cancer cells in a tissue sample.

In accordance with the present invention, a suitable single dose size is a dose that results in elicitation of an immune response in a patient when administered one or more times over a suitable time period. Doses can vary depending upon the disease being treated or against which a vaccine is directed. For example, in the treatment of cancer, a suitable single dose can be dependent upon whether the cancer being treated is a primary tumor or a metastatic form of cancer. One of skill in the art can readily determine appropriate single dose sizes for a given patient based on the size of a patient and the route of administration. One of skill in the art can monitor the effectiveness of the immunization or treatment by measuring, for example, proliferative response of immune effector cells, upregulation of immune activation markers on cells, cytokine responses, cytotoxicity, antibody production, by enumerating antigen-specific T cells, or monitoring delayed type hypersensitivity (DTH) responses.

In one aspect of the invention, a suitable single dose of a vaccine or therapeutic composition of the present invention to elicit an immune response in a patient is an amount that, when administered by any route of administration, elicits a cellular and/or humoral immune response in vivo in a patient, as compared to a patient which has not been administered with the therapeutic composition of the present invention (i.e., a control patient), as compared to the patient prior to administration of the vaccine or composition, or as compared to a standard established for the particular disease, patient type and composition.

A suitable single dose of a vaccine or therapeutic composition to elicit an immune response against a tumor is an amount that is sufficient to reduce, stop the growth of, and preferably eliminate, the tumor following administration of the composition into the tissue of the patient that has cancer. A single dose of a vaccine or therapeutic composition useful to elicit an immune response against an infectious disease and/or against a lesion associated with such a disease is substantially similar to those doses used to treat a tumor, wherein the amount is sufficient to reduce, eliminate, or prevent at least one symptom of an infectious disease or lesion associated with such disease. Similarly, a single dose of a therapeutic composition useful to elicit an immune response against an allergen is substantially similar to those doses used to treat a tumor, wherein the amount is sufficient to reduce, eliminate or prevent at least one symptom of allergic inflammation.

It will be obvious to one of skill in the art that the number of doses administered to a patient is dependent upon the extent of the disease and the response of an individual patient to the treatment. For example, a large tumor may require more doses than a smaller tumor. In some cases, however, a patient having a large tumor may require fewer doses than a patient with a smaller tumor, if the patient with the large tumor responds more favorably to the therapeutic composition than the patient with the smaller tumor. In addition, immunizations typically require an initial administration of the vaccine, followed by one or more boosters to enhance immunization against an antigen. Thus, it is within the scope of the present invention that a suitable number of doses includes any number required to treat a given disease.

As discussed above, a vaccine or therapeutic composition of the present invention is administered to a patient in a manner effective to deliver the composition to a cell, a tissue, and/or systemically to the patient, whereby elicitation of an antigen-specific immune response is achieved as a result of the administration of the vaccine or composition. Suitable administration protocols include any in vivo or ex vivo administration protocol. The preferred routes of administration will be apparent to those of skill in the art, depending on the type of condition to be prevented or treated; whether the vaccine or composition is nucleic acid based, protein based, or cell based; the antigen used; and/or the target cell/tissue. For proteins or nucleic acid molecules, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. In one embodiment, routes of delivery which elicit an immune response in the mucosal tissues are preferred. Such routes include bronchial, intradermal, intramuscular, intranasal, other inhalatory, rectal, subcutaneous, topical, transdermal, vaginal and urethral routes. Some particularly preferred routes of administration include, intravenous, intraperitoneal, subcutaneous, intradermal, intranodal, intramuscular, transdermal, inhaled, intranasal, rectal, vaginal, urethral, topical, oral, intraocular, intraarticular, intracranial, and intraspinal. Combinations of routes of delivery can be used and in some instances, may enhance the therapeutic effects of the vaccine or composition.

Ex vivo administration refers to performing part of the regulatory step outside of the patient, such as administering a composition (nucleic acid or protein) of the present invention to a population of cells removed from a patient under conditions such that the composition contacts and/or enters the cell, and returning the cells to the patient. Ex vivo methods are particularly suitable when the target cell can easily be removed from and returned to the patient.

Many of the above-described routes of administration, including intravenous, intraperitoneal, intradermal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a composition to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety.

For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. In addition, Liu et al., Nature Biotechnology 15:167, 1997, demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y. Acad. Sci. 811:299-308; and von der Leyen et al., 1995, Proc Natl Acad Sci USA 92:1137-1141).

Delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al. (1999, J. Clin. Invest. 104:21-29) demonstrated that an adenoviral vector encoding a β2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572).

In the area of neuronal gene delivery, multiple successful in vivo gene transfers have been reported. Millecamps et al. reported the targeting of adenoviral vectors to neurons using neuron restrictive enhancer elements placed upstream of the promoter for the transgene (phosphoglycerate promoter). Such vectors were administered to mice and rats intramuscularly and intracerebrally, respectively, resulting in successful neuronal-specific transfection and expression of the transgene in vivo (Millecamps et al., 1999, Nat. Biotechnol. 17:865-869). As discussed above, Bennett et al. reported the use of adeno-associated viral vector to deliver and express a gene by subretinal injection in the neural retina in vivo for greater than 1 year (Bennett, 1999, ibid.).

Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti- T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include an isolated nucleic acid molecule of the present invention, and preferably includes a recombinant molecule of the present invention that preferably is replication, or otherwise amplification, competent. A naked nucleic acid reagent of the present invention can comprise one or more nucleic acid molecules of the present invention including a bicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

In the method of the present invention, vaccines and therapeutic compositions can be administered to any member of the Vertebrate class, Mammalia, including, without limitation, primates, rodents, livestock and domestic pets. Livestock include mammals to be consumed or that produce useful products (e.g., sheep for wool production). Preferred mammals to protect include humans, dogs, cats, mice, rats, sheep, cattle, horses and pigs, with humans and dogs being particularly preferred, and humans being most preferred.

A therapeutic composition or vaccine of the present invention which includes an antigen (or encodes an antigen, in the case of a nucleic acid based composition) that elicits an immune response against a tumor is useful for eliciting an immune response in a patient that has cancer, including both primary tumors and metastatic forms of cancer. A therapeutic composition of the present invention is advantageous in that the composition overcomes the mechanisms by which cancer cells avoid immune elimination (i.e., by which cancer cells avoid the immune response effected by the mammal in response to the disease). Cancer cells can avoid immune elimination by, for example, being only slightly immunogenic, modulating cell surface antigens and inducing immune suppression. The effects of the mutated E1A protein of the present invention with regard to tumor cells has been discussed in detail elsewhere herein.

Cancers to be treated or prevented using the method and composition of the present invention include, but are not limited to, melanomas, squamous cell carcinoma, breast cancers, cervical cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof. Preferably, expression of the tumor antigen in a tissue of a patient that has cancer in conjunction with the expression of the mutated E1A protein produces a result selected from the group of alleviation of the cancer, reduction of a tumor associated with the cancer, elimination of a tumor associated with the cancer, prevention of metastatic cancer, prevention of the cancer and stimulation of effector cell immunity against the cancer.

A vaccine or therapeutic composition of the present invention which includes an antigen that is associated with an infectious disease (e.g., from a pathogenic microorganism that causes the disease) is advantageous for eliciting an immune response in a patient who has an infectious disease and who will benefit from an immune response or to elicit an immune response in a patient that will protect the patient against subsequent infection by the pathogen. Preferred infectious diseases to treat or against which to vaccinate are diseases caused by a pathogen in which the elicitation of an immune response against the pathogen can result in a prophylactic or therapeutic effect as previously described herein. Such a method provides a long term, targeted therapy for primary lesions (e.g., granulomas) resulting from the propagation of a pathogen. As used herein, the term “lesion” refers to a lesion formed by infection of a patient with a pathogen. The infectious disease can be either chronic or acute. Preferably, delivery of the antigen to a tissue of a patient that has an infectious disease produces a result which includes alleviation of the disease, regression of established lesions associated with the disease, alleviation of symptoms of the disease, immunization against the disease and/or stimulation of effector cell immunity against the disease.

Pathogens from which immunogens useful in the present invention can be derived include, but are not limited to, bacteria (including intracellular bacteria which reside in host cells), viruses, parasites (including internal parasites), fungi (including pathogenic fungi), endoparasites, ectoparasites, and prions (e.g., bovine spongiform encephalopathy; BSE). Preferred infectious diseases to treat or prevent with a vaccine or therapeutic composition of the present invention include chronic infectious diseases, including pulmonary infectious diseases, such as tuberculosis. Particularly preferred infectious diseases to treat with a therapeutic composition of the present invention include viral infections such as those caused by human immunodeficiency virus, Epstein Barr virus, other Herpes viruses, papilloma virus, and Hepatitis viruses; mycobacterial infections such as that caused by Mycobacterium tuberculosis, and ; fungal infections such as those caused by Candida, Blastomyces, and Histoplasma; parasitic infections such as those caused by Toxoplasma; and bacterial infectious diseases such as those caused by Cryptococcus, Bacillus anthracis and Yersenia pestis, Staphylococcus, Pseudomonas, Streptococcus, Enterococcus, Salmonella, Pasteurella, Fransicella and other gram negative and gram positive bacterial pathogens.

A therapeutic composition of the present invention which includes an antigen that elicits an immune response against an allergen is advantageous for eliciting an immune response in a patient that has or is at risk of developing a disease associated with allergic inflammation. A disease associated with allergic inflammation is a disease in which the elicitation of one type of immune response (e.g., a Th2-type immune response) against a sensitizing agent, such as-an allergen, can result in the release of inflammatory mediators that recruit cells involved in inflammation in a patient, the presence of which can lead to tissue damage and sometimes death. Using the present invention, one can elicit a Th1-type response against an allergen, which can have prophylactic or therapeutic effects such that allergic inflammation is alleviated or reduced. A therapeutic composition for use in the elicitation of an immune response in a patient that has a disease associated with allergic inflammation comprises an immunogen that is an allergen, an immunogenic portion of an allergen, an epitope of an allergen, or combinations thereof. Preferably, delivery of the allergen to a tissue of a patient that has a disease associated with allergic inflammation or is at risk of developing the disease, produces a result which includes alleviation of the disease, alleviation of symptoms of the disease, desensitization against the disease and stimulation a protective immune response against the disease. Preferred diseases associated with allergic inflammation which are preferable to treat using the method and composition of the present invention include, allergic airway diseases, allergic rhinitis, allergic conjunctivitis and food allergy.

The following examples are provided for the purpose of illustration and are not intended to limit the scope of the present invention.

### EXAMPLES

### Example 1

The following example demonstrates that tumor cells that express a mutant form of E1A that does not bind p300/CBP (E1AΔp300) are highly tumorigenic and resistant to NK cell killing.

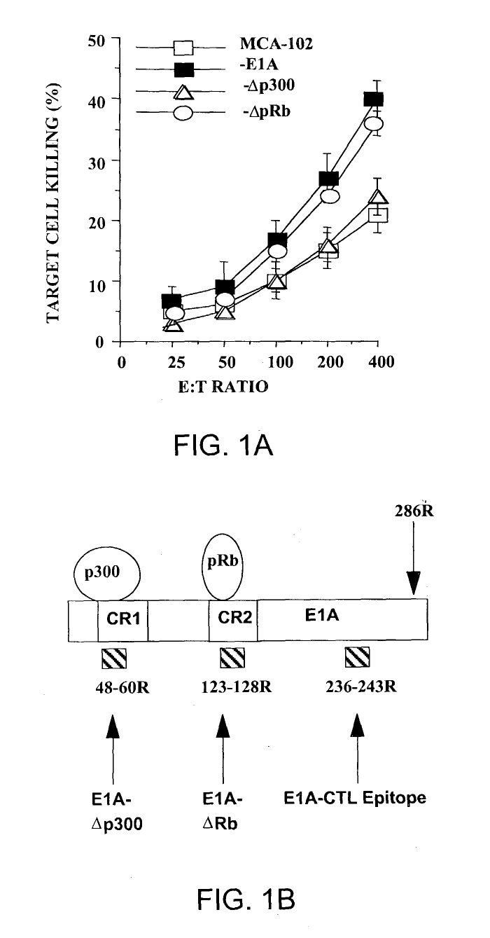

In this experiment, two tumor cell lines were transfected with a nucleic acid construct that encode mutated E1A proteins that abolish binding to p300/CBP or pRb, respectively (FIG. 1B), but which retain the immunodominant CTL epitope (residues 236-243 of SEQ ID NO:2 in B6 mice; see FIG. 1B). E1A-Δp300 encodes a mutated adenovirus serotype 5 E1A protein that has a deletion of amino acids at positions 48-60 (positions 48-60 of SEQ ID NO:2). E1A-ΔpRb encodes a mutated adenovirus serotype 5 E1A protein that has a deletion of amino acids at positions 123-128 (positions 123-128 of SEQ ID NO:2). One of the tumor cell lines used was MCA-102, which is highly oncogenic and which was chosen because unlike many murine tumor lines, MCA-102 cells are non-immunogenic, even when transduced by the costimulatory molecules B7.1 or B7.2 (Chen et al., 1994; Mule et al., 1987; Yang et al., 1995). The second tumor cell line used was the weakly immunogenic tumor cell line, MCA-205. The transfected tumor cells were evaluated for their ability to induce NK sensitivity and for their ability to induce tumors in mice that were: immunologically intact, lacked T cells (nude mice) or lacked both NK and T cells (i.e., CD3-epsilon-transgenic mice). The results are illustrated in Table 1 and in FIG. 1A.

Unlike wild-type E1A or E1A-ΔpRb, E1A-Δp300 was unable to induce NK sensitivity (FIG. 1A) against tumor cells expressing the construct. Tumor-induction studies showed that in contrast, MCA-E1A-ΔpRb cells and MCA-E1A-Δp300 cells exhibit equivalent tumorigenicity as compared to the parental (untransfected) or MCA-E7 cells (transfected with the wild-type E1A) (Table 1).

TABLE 1

| | TPD50 | | TPD50 | Class 1 MH | | |
|---|---|---|---|---|---|---|
| | Normal | TPD50 | CD3-E-tg | Antigen | NK cell | Induction |
| Cell line | mice | Nude mice | mice | Expression | Lysis | of CTL |
| MCA-102 | 2.5-3.5 | 2-2.5 | 1.75 | 100% | No | No |
| E1A-CL1 | 7-7.5 | 5 | 2 | 120% | Yes | Yes |
| E1A-CL2 | 7-7.5 | 5 | 2 | 400% | Yes | Yes |
| E7-CL1 | 2.5-3.5 | 2-2.5 | 2 | 200% | No | No |
| E7-CL2 | 2.5-3.5 | 2-2.5 | 2 | 200% | No | No |
| E7/E6 | 2.5-3.5 | ND | ND | 220% | No | No |
| E1A&E7 | 7-7.5 | ND | ND | 290% | No | ND |
| E1A-Δp300 | 2.5-3.5 | ND | ND | 200% | No | No |

### Example 2

The following example demonstrates that macrophages kill E1A, and not E1A-Δ-p300 or E7-expressing tumor cells, and that killing is predominately nitric oxide-dependent and caspase dependent.

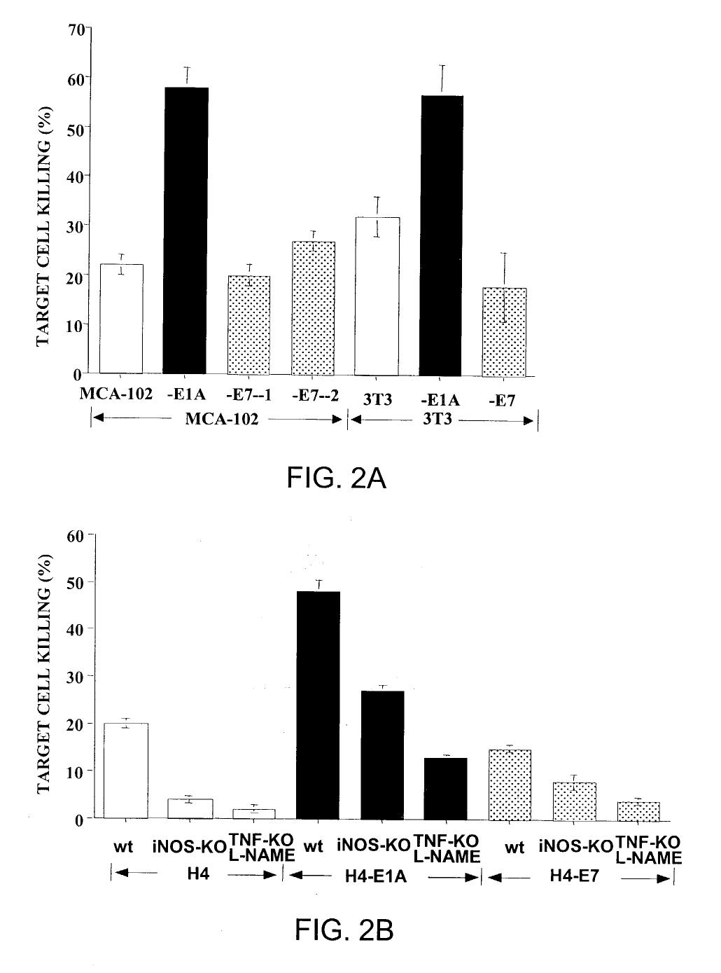

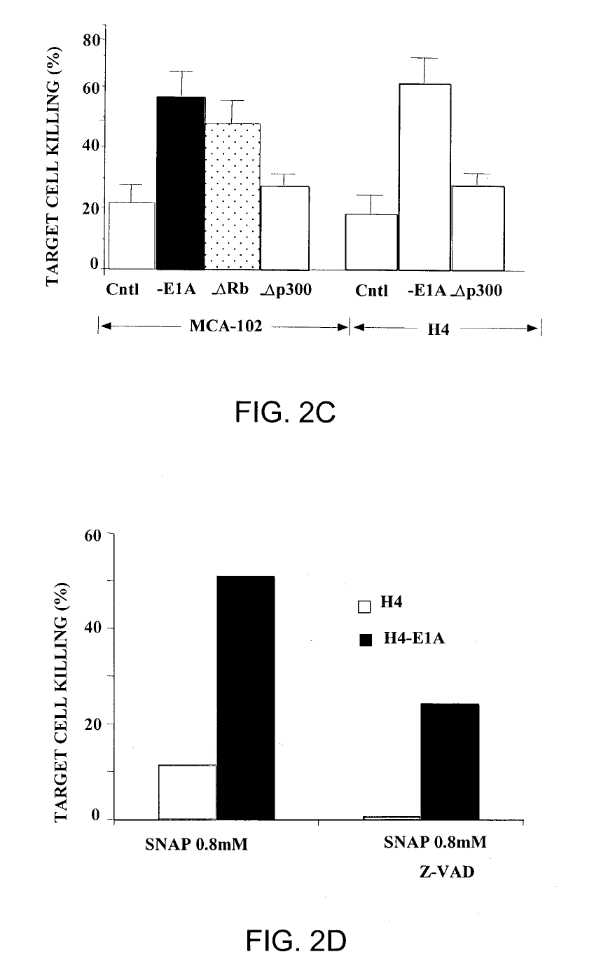

The present inventor compared the ability of E1A or E7 expression to sensitize tumor cells to killing by macrophages. Through the use of macrophages derived from mice with specific genetic deficiencies, the contribution of TNF-β, nitric oxide (NO), fas-ligand (fas-L) and superoxide in the lysis of E1A- or E7-expressing cells (E7 is from human papilloma virus HPV16) by activated macrophages was determined. Results showed that E1A-, but not HPV16-E7-expression, sensitized cells to lysis by activated macrophages (FIG. 2A). Activated macrophages predominately utilized NO and to a lesser extent, TNF-α, but did not utilize fas-L or superoxide to kill E1A-expressing cells (FIG. 2B, data not shown on gld or phox91-/- mice). These data are consistent with recent observations by others showing that unlike E1A-expressing cells (Duerksen-Hughes, et al., 1989, J. Immunol. 143:4193; Chen et al., 1988, In Monokines and Other Non-lymphocyte Cytokines, N. C. Powanda, ed., p. 243; Cook et al., 1989, J Immunol 142:4527), E7-expressing cells are resistant to lysis by TNF-α (Thompson et al., 2001, Oncogene 20:3629; Basile et al., 2001, J Biol Chem 276:22522).

Similar to the present inventor's results on NK cell killing, E1A-Δp300-expression was unable to induce sensitivity to macrophage killing (FIG. 2C). Next, by incubating cells in the presence of of SNAP (S-Nitroso-N-acetylpnicillamine), an NO donor, it was shown that E1A-sensitized tumor cells to NO, in the absence of macrophages (FIG. 2D. Furthermore, NO-dependent killing of E1A-expressing H4 cells(a human fibrosarcoma cell line) was significantly, but not totally, inhibited by z-VAD-FMK, a caspase inhibitor (FIG. 2D). This latter observation is consistent with prior reports indicating that NO can kill tumor cells through caspase-dependent and independent means. The demonstration that E1A sensitizes cells to NO-dependent killing has not been previously reported, to the present inventor's knowledge.

### Example 3

The following example shows that E1A-sensitizes cells to killing by both NK cells and activated macrophages.

Since NKG2D activating receptors are expressed on both NK cells and macrophages, the present inventor speculated that E1A may upregulate NKG2D ligands. To test this hypothesis, a variety of control and E1A-transfected, human and murine cell lines were screened with NKG2D-Ig Fc-fusion protein. E1A-expression, but not E1A-Δ-p300- or E7-expression (data not shown), consistently upregulated NKG2D ligands in both murine and human cells (data not shown). Subsequent studies utilizing real time PCR and northern analysis established that the NKG2D ligand that was induced was Rae-167 . Additional studies by the present inventor demonstrated that E1A upregulates several NKG2D ligands in human cells which include MICA/B, ULBP-2 and ULBP-3 (data not shown). This is the first viral oncogene that has been shown to upregulate ligands for activating NK cell receptors.

### Example 4

The following example demonstrates that E1A, but not E1A-Δ-p300, upregulates Hsp-70.

The expression of Hsp70 in cells induces resistance to killing by NO, TNF-α and activated macrophages (Klein et al., 2002, Biochem J 362:635; Gurbuxani et al., 2001, Oncogene 20:7478; Burkart et al., 2000, J Biol Chem 275:19521; Melcher et al., 1998, Nat Med 4:581; Bellmann et al., 1996, FEBS Lett 391:185; Jaattela et al., 1992, EMBO Journal 11:3507). Expression of E1A in cells increases production of Hsp70 (Nevins, 1982, Cell 29:913; White et al., 1988, J. Virol. 62:4153).

The present inventors investigated the possibility that the Hsp70-induced resistance to killing by NO, TNF-α and activated macrophages is blocked by the expression of E1A. Therefore, expression of Hsp70 protein was measured in MCA-102, MCA-102-E1A, H4, H4-E1A and H4-E7 cells by western analysis. Briefly, for the quantitation of Hsp70, 60 mm plates of cells (MCA-102, MCA-E1A, H4, H4-E1A and H4-E7) were lysed in RIPA lysis buffer (1% NP-40, 50 mM Tris pH 8.0, 150 mM NaCl, 0.55% Sodium Deoxycholate, 0.1% SDS). Protein concentrations were measured using Biorad Protein Assay (Biorad, Hercules Calif.). Ten μg of protein was separated on a 4-16% SDS PAGE gel. Proteins were then electrophoretically transferred onto a PVDF membrane (Biorad, Hercules, Calif.). The membrane was blocked with 5% nonfat milk /PBST and incubated for 1 hour with a monoclonal antibody that recognizes only the inducible form of Hsp-70 (Santa Cruz Biotechnology, Santa Cruz, Calif.). After several washes in PBST, the blot was then incubated for 1 h with HRP-conjugated anti-mouse antibody (Amersham, Piscataway, N.J.). The Hsp70 protein was visualized as per manufacturer's instructions using the Renaissance Chemiluminescence Kit (DuPont-NEN, Boston, Mass.).

Results showed that, in contrast to E7, the amount of Hsp 70 was increased in E1A-expressing cells (data not shown). These data indicated that E1A mutes the anti-apoptotic function of Hsp70 in tumor cells. In a further experiment, the present inventor determined by both Western Blot analysis and gene array analysis that a mutant form of E1A that does not interact with p300 (E1A-Δ-p300) does not upregulate Hsp70 while the wild-type E1A upregulates Hsp70 in both analyses (data not shown).

Therefore, in agreement with prior studies, the present inventors show that E1A upregulates the expression of Hsp70 on both murine and human tumor cells. Hsp70 can increase the immunogenicity of endogenously expressed tumor antigens and enhance immune-mediated tumor rejection. Several mechanisms likely contribute to this anti-tumor activity of heat shock proteins including the ability to chaperone antigenic peptides to APC, to induce the maturation of dendritic cells, to stimulate dendritic cells and macrophages to produce proinflammatory cytokines, chemokines and NO, and to enhance tumor cell killing by NK cells (Multhoff et al., 1997, J Immunol. 158:4341; Srivastava, 2002, Annu Rev Immunol. 2002:395; Panjwani et al, 2002, J Immunol. 168:2997; Todryk et al., 1999, J Immunol 163:1398; Basu et al., 2001, Immunity 14:303; and Binder et al., 2001, J Biol Chem. 276:17163). Hsp70 also protects tumor cells against a variety of apoptotic-inducing stimuli, including TNFα and NO (Klein et al., 2002, Biochem J 362:635; Gurbuxani et al., 2001, Oncogene 20:7478; Burkart et al., 2000, J Biol Chem 275:19521; Melcher et al., 1998, Nat Med 4:581; Bellmann et al., 1996, FEBS Lett 391:185). For example, over expression of Hsp70 in tumor cells decreases their sensitivity to macrophage-induced killing while increasing their tumorigenicity in syngeneic hosts (Gurbuxani et al., 2001, supra). However, the present inventors' data shows that E1A abrogates the ability of Hsp70 to protect tumor cells from killing by NK cells and macrophages. The capacity of E1A to abrogate the anti-apoptotic, tumor-protective effects of Hsp70 while at the same time harnessing the pro-immunogenic effects of Hsp70 has not been previously appreciated.

### Example 5

The following example demonstrates that MCA tumor cells that express a mutant form of E1A that does not bind p300/CBP (E1AΔp300) inefficiently induce E1A-specific CTL (E1A-CTL).

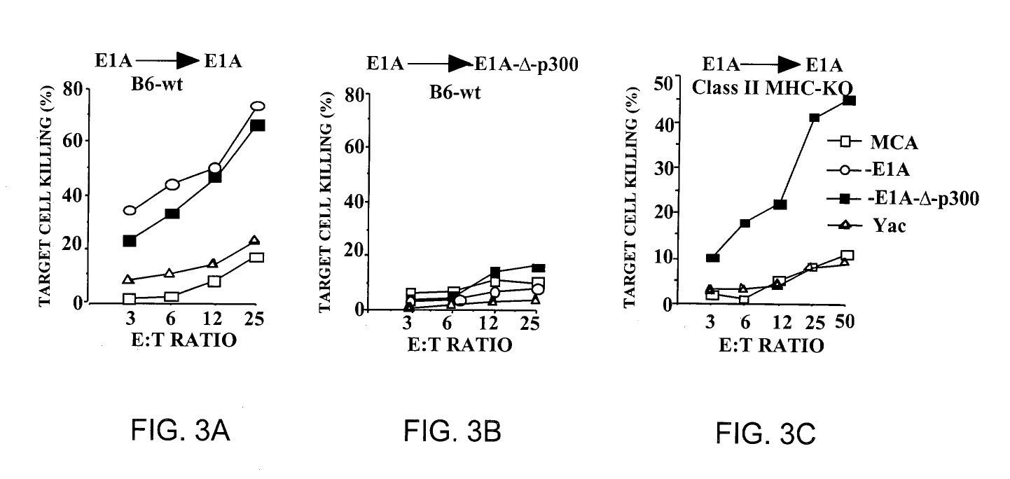

In experiments discussed above, the inventor showed that MCA-102-E1AΔp300 cells are resistant to NK lysis and fail to induce either Rae-1δ or Hsp70. In this experiment, the ability of the various constructs to generate cytotoxic T lymphocyte (CTL) responses was investigated. Briefly, one million, live MCA-102-E1A cells (FIGS. 3A and 3C) or MCA-102-E1A-Δp300 cells (FIG. 3B) were injected intraperitoneally into: (1) C57BL/6mice (FIGS. 3A and 3B); or (2) class II MHC knockout (KO) mice (FIG. 3C). Two weeks post-injection, splenocytes were harvested and a five day, in vitro priming step was performed using irradiated MCA-102-E1A cells as stimulator cells. Cytotoxicity assays were performed using various target cells as shown in FIGS. 3A-3C.

FIG. 3A shows that E1A-CTL (i.e., cytotoxic T lymphocytes that recognize E1A) are generated in normal mice immunized with E1A and specifically kill both E1A-expressing target cells (open circles) and MCA-102-E1A-Δp300-expressing target cells (closed squares). However, as shown in FIG. 3B, injection of mice with MCA-E1A-Δp300failed to induce CTL that could kill E1A-expressing target cells or MCA-102-E1A-Δp300-expressing cells.

The inability of E1A-Δp300 to generate E1A-CTL could be due to a deletion of the CD4⁺ T helper cell epitope. Therefore, the inventor determined if the generation of E1A-CTL requires class II-restricted, CD4+ T cells through the use of class II-KO mice (FIG. 3C). Class II-KO mice were injected with MCA-102-E1A cells and E1A-CTL were generated as described. As shown in FIG. 3C, E1A-CTL were efficiently generated in class II-KO mice. Because E1A-CTL generation occurred in the absence of class II restricted, CD4⁺ helper T cells, the possible deletion of a T helper epitope in E1A-Δp300does not account for their inability to generate E1A-CTL. Therefore, these results demonstrated that Class II-restricted, CD4+ T cells are not required for the generation of E1A-CTL. The ability of MCA-102-E1A or E1A-Δp300 cells to generate or be recognized by antigen-specific CTL was independent of their respective, surface class I levels (data not shown).

### Example 6

The following example demonstrates that expression of a nucleic acid molecule encoding a mutated E1A protein-antigen chimera sensitized tumor cells to lysis by activated macrophages.

A nucleic acid molecule was constructed which encoded amino acid positions 1-114 of adenovirus serotype 5 E1A (positions 1-114 of SEQ ID NO:2) fused to amino acids 14-98 of HPV type 16 E7 (in this construct, the E7 molecule is the antigen). As discussed in Example 2, expression of E1A, but not E7 sensitized tumor cells to lysis by activated macrophages, and these macrophages utilized nitric oxide (NO) and TNF-α, but not Fas-L or superoxide generation, to kill the E1A-expressing tumor cells. In this experiment, expression of the mutated E1A-E7 chimera sensitized the tumor cells to killing by macrophages. Therefore, these experiments demonstrate that E1A (including mutated E1A) is a highly modular protein whose biological properties are not altered by linking it with other antigens (including a viral oncoprotein).

### Example 7

The following example demonstrates that E1A not only inhibits baseline levels of TGF-β, but also inhibits levels induced following interaction with the phosphatidylserine receptor (PtdSerR), which recognizes apoptotic cells.

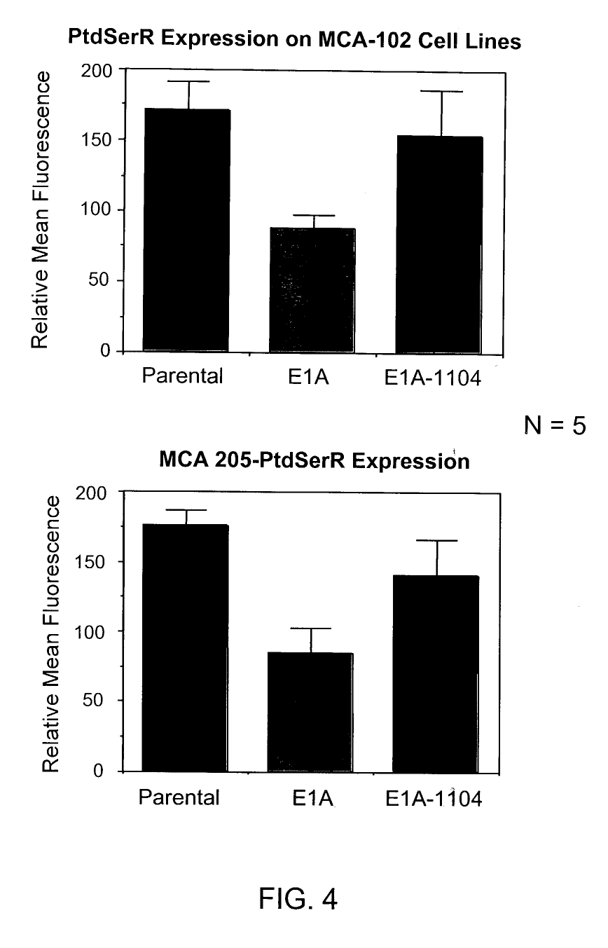

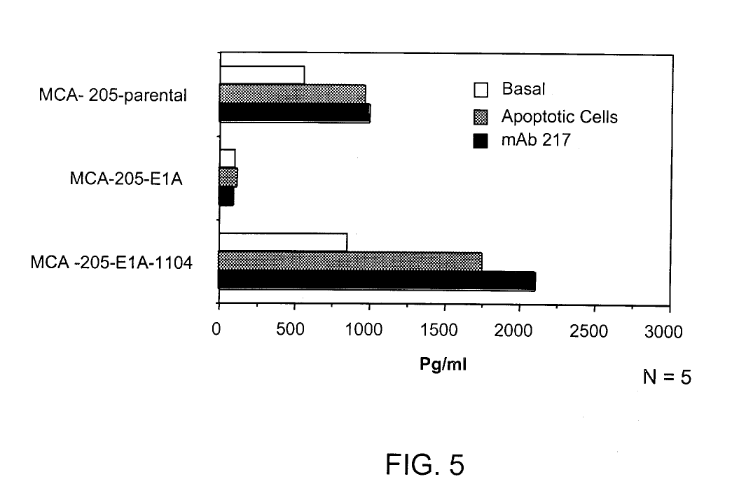

As shown in FIG. 4, expression of E1A (E1A), but not a mutant form of E1A that does not interact with p300/CBP (E1A-1104), reduces the expression of phosphatidylserine receptor (PtdSerR) by two different tumor cell lines. FIG. 5 shows that expression of E1A (MCA-205-E1A), but not a mutant form of E1A that does not interact with p300/CBP (MCA-205-E1A-1104), decreases the production of transforming growth factor β (TGFβ) by tumor cells (both the baseline TGFβ production and the TGFβ production that is induced following stimulation of the phosphatidylserine receptor (mAb217) or apoptotic cells).

All publications and references disclosed herein are incorporated by reference in their entireties.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated nucleic acid molecule, comprising:
a) a first nucleic acid sequence encoding an antigen; and
b) a second nucleic acid sequence encoding a mutated Group C adenoviral E1A protein;
wherein the amino acid sequence of the mutated E1A protein differs from the amino acid sequence of a wild-type E1A protein by at least one mutation, wherein said at least one mutation comprises a mutation in the CR2 region of E1A that abolishes the ability of the mutated E1A protein to bind to pRb;
wherein the mutated E1A protein retains an amino acid sequence from the wild-type E1A protein that binds to p300/CBP; and
wherein the mutated E1A protein retains an amino acid sequence from the wild-type E1A protein that binds to the C-terminal binding protein (CtBP).

2. The isolated nucleic acid molecule of claim 1, wherein the amino acid sequence of the mutated E1A protein further differs from the amino acid sequence of the wild-type E1A protein by a deletion of from 1 to about 5 amino acid residues from the C-terminal end of the wild-type E1A amino acid sequence, wherein the mutated E1A protein has antioncogenic activity and is immunogenic.

3. The isolated nucleic acid molecule of claim 1, wherein the mutated E1A protein differs from the wild-type E1A protein by a deletion of amino acids in the CR2 region that abolishes the ability of the mutated E1A protein to bind to pRb.

4. The isolated nucleic acid molecule of claim 1, wherein the mutated E1A protein differs from the wild-type E1A protein by a substitution or insertion of amino acids in the CR2 region that abolishes the ability of the mutated E1A protein to bind to pRb.

5. The isolated nucleic acid molecule of claim 1, wherein the mutated E1A protein is identical to the wild-type E1A protein by retention of amino acid residues in the CR1 region and amino acid residues in the first 10 residues from the N-terminus of the protein, wherein said retained residues bind to p300/CBP.

6. The isolated nucleic acid molecule of claim 1, wherein the mutated E1A protein is identical to the wild-type E1A protein by retention of amino acid residues in the first 60 residues from the N-terminus of the protein, wherein said retained residues bind to p300/CBP.

7. The isolated nucleic acid molecule of claim 1, wherein the mutated E1A protein is identical to the wild-type E1A protein by retention of amino acid residues in the CR3 region.

8. The isolated nucleic acid molecule of claim 1, wherein the mutated E1A protein is identical to the wild-type E1A protein by retention of amino acid residues in the last 44 amino acid positions from the C-terminus.

9. The isolated nucleic acid molecule of claim 1, wherein the mutated E1A sensitizes cells containing said mutated E1A to killing by immune effector cells.

10. The isolated nucleic acid molecule of claim 1, wherein the mutated E1A enhances upregulation of Hsp70 by cells containing said mutated E1A.

11. The isolated nucleic acid molecule of claim 1, wherein the wild-type E1A is selected from the group consisting of an adenovirus serotype 5 13S E1A protein represented by SEQ ID NO:2, an adenovirus serotype 2 13S E1A protein represented by SEQ ID NO:6, an adenovirus serotype 5 12S E1A protein represented by SEQ ID NO:4, and an adenovirus serotype 2 12S E1A protein represented by SEQ ID NO:8.

12. The isolated nucleic acid molecule of claim 11, wherein the amino acid sequence of the mutated E1A protein further differs from the amino acid sequence of the wild-type E1A protein by a deletion or substitution of at least one amino acid residue within the amino acid sequence between:
a) about position 189 and about position 270 of SEQ ID NO:2;
b) about position 189 and about position 270 of SEQ ID NO:6;
c) about position 133 and about position 224 of SEQ ID NO:4; or
d) about position 133 and about position 224 of SEQ ID NO:8;
wherein the mutated E1A protein has antioncogenic activity and is immunogenic.

13. The isolated nucleic acid molecule of claim 11, wherein the amino acid sequence of the mutated E1A protein further differs from the amino acid sequence of the wild-type E1A protein by a deletion or substitution of at least one amino acid residue within the amino acid sequence between:
a) about position 189 and about position 245 of SEQ ID NO:2;
b) about position 189 and about position 245 of SEQ ID NO:6;
c) about position 133 and about position 199 of SEQ ID NO:4; or
d) about position 133 and about position 199 of SEQ ID NO:8;
wherein the mutated E1A protein has anti-oncogenic activity and is immunogenic.

14. The isolated nucleic acid molecule of claim 11, wherein the amino acid sequence of the mutated E1A protein further differs from the amino acid sequence of the wild-type E1A protein by a deletion or substitution of at least 10 amino acid residues within the amino acid sequence between:
a) about position 189 and about position 270 of SEQ ID NO:2;
b) about position 189 and about position 270 of SEQ ID NO:6;
c) about position 133 and about position 224 of SEQ ID NO:4; or
d) about position 133 and about position 224 of SEQ ID NO:8;
wherein the mutated E1A protein has anti-oncogenic activity and is immunogenic.

15. The isolated nucleic acid molecule of claim 11, wherein the amino acid sequence of the mutated E1A protein further differs from the amino acid sequence of the wild-type E1A protein by a deletion or substitution of at least 10 amino acid residues within the amino acid sequence between:
a) about position 189 and about position 245 of SEQ ID NO:2;
b) about position 189 and about position 245 of SEQ ID NO:6;
c) about position 133 and about position 199 of SEQ ID NO:4; or
d) about position 133 and about position 199 of SEQ ID NO:8;
wherein the mutated E1A protein has anti-oncogenic activity and is immunogenic.

16. The isolated nucleic acid molecule of claim 11, wherein the amino acid sequence of the mutated E1A protein further differs from the amino acid sequence of the wild-type E1A protein by a deletion or substitution of at least 20 amino acid residues within the amino acid sequence between:
a) about position 189 and about position 270 of SEQ ID NO:2;
b) about position 189 and about position 270 of SEQ ID NO:6;
c) about position 133 and about position 224 of SEQ ID NO:4; or
d) about position 133 and about position 224 of SEQ ID NO:8;
wherein the mutated E1A protein has anti-oncogenic activity and is immunogenic.

17. The isolated nucleic acid molecule of claim 11, wherein the amino acid sequence of the mutated E1A protein further differs from the amino acid sequence of the wild-type E1A protein by a deletion or substitution of at least 20 amino acid residues within the amino acid sequence between:
a) about position 189 and about position 245 of SEQ ID NO:2;
b) about position 189 and about position 245 of SEQ ID NO:6;
c) about position 133 and about position 199 of SEQ ID NO:4; or
d) about position 133 and about position 199 of SEQ ID NO:8;
wherein the mutated E1A protein has anti-oncogenic activity and is immunogenic.

18. The isolated nucleic acid molecule of claim 11, wherein the amino acid sequence of the mutated E1A protein is identical to the amino acid sequence of the wild-type protein by retention of amino acid positions:
a) from about 143 to about 188 of SEQ ID NO:2; or
b) from about 143 to about 188 of SEQ ID NO:6.

19. The isolated nucleic acid molecule of claim 11, wherein the amino acid sequence of the mutated E1A protein differs from the amino acid sequence of the wild-type E1A protein by a mutation in at least amino acid positions:
a) 124-127 of SEQ ID NO:2;
b) 124-127 of SEQ ID NO:6;
c) 124-127 of SEQ ID NO:4; or
d) 124-127 of SEQ ID NO:8;
wherein the mutation is sufficient to abolish the ability of the mutated E1A protein to bind to pRb.

20. The isolated nucleic acid molecule of claim 19, wherein the mutation is a deletion of said amino acid positions.

21. The isolated nucleic acid molecule of claim 11, wherein the amino acid sequence of the mutated E1A protein differs from the amino acid sequence of the wild-type E1A protein by a mutation in at least amino acid positions:
a) 120-140 of SEQ ID NO:2;
b) 120-140 of SEQ ID NO:6;
c) 120-132 of SEQ ID NO:4; or
d) 120-132 of SEQ ID NO:8;
wherein the mutation is sufficient to abolish the ability of the mutated E1A protein to bind to pRb.

22. The isolated nucleic acid molecule of claim 21, wherein said mutation is a deletion of said amino acid positions.

23. The isolated nucleic acid molecule of claim 11, wherein the amino acid sequence of the mutated E1A protein is identical to SEQ ID NO:2, SEQ ID NO:6, SEQ ID NO:4 or SEQ ID NO:8 by retention of amino acids in at least positions 1-10 and from about positions 48 to about position 60;
wherein the mutated E1A binds to p300/CBP.

24. The isolated nucleic acid molecule of claim 11, wherein the amino acid sequence of the mutated E1A protein is identical to SEQ ID NO:2, SEQ ID NO:6, SEQ ID NO:4 or SEQ ID NO:8 by retention of amino acids in at least positions 1-40 and from about positions 48 to about position 60;
wherein the mutated E1A binds to p300/CBP.

25. The isolated nucleic acid molecule of claim 11, wherein the amino acid sequence of the mutated E1A protein is identical to SEQ ID NO:2, SEQ ID NO:6, SEQ ID NO:4 or SEQ ID NO:8 by retention of amino acids in at least positions 1-60;
wherein the mutated E1A binds to p300/CBP.

26. The isolated nucleic acid molecule of claim 1, wherein the mutated E1A comprises, in consecutive order, amino acid positions 1 to about 140 and amino acid positions from about 271 to about 284 of SEQ ID NO:2 or SEQ ID NO:6, wherein amino acids in the CR2 region have been mutated to abolish the ability of the mutated E1A to bind to pRb.

27. The isolated nucleic acid molecule of claim 1, wherein the mutated E1A comprises, in consecutive order, amino acid positions 1 to about 188 and amino acid positions from about 271 to about 284 of SEQ ID NO:2 or SEQ ID NO:6, wherein amino acids in the CR2 region have been mutated to abolish the ability of the mutated E1A to bind to pRb.

28. The isolated nucleic acid molecule of claim 1, wherein the mutated E1A comprises, in consecutive order, amino acids 1 to about 123, amino acids from about 128 to about 140, and amino acids from about 271 to about 284, of SEQ ID NO:2 or SEQ ID NO:6.

29. The isolated nucleic acid molecule of claim 1, wherein the mutated E1A comprises, in consecutive order, amino acids 1 to about 123, amino acids from about 128 to about 188, and amino acids from about 271 to about 284, of SEQ ID NO:2 or SEQ ID NO:6.

30. The isolated nucleic acid molecule of claim 1, wherein the mutated E1A comprises, in consecutive order, amino acid positions 1 to about 140 and amino acid positions from about 246 to about 289 of SEQ ID NO:2 or SEQ ID NO:6, wherein amino acids in the CR2 region have been mutated to abolish the ability of the mutated E1A to bind to pRb.

31. The isolated nucleic acid molecule of claim 1, wherein the mutated E1A comprises, in consecutive order, amino acid positions 1 to about 188 and amino acid positions from about 246 to about 289 of SEQ ID NO:2 or SEQ ID NO:6, wherein amino acids in the CR2 region have been mutated to abolish the ability of the mutated E1A to bind to pRb.

32. The isolated nucleic acid molecule of claim 1, wherein the mutated E1A comprises, in consecutive order, amino acids 1 to about 123, amino acids from about 128 to about 140, and amino acids from about 246 to about 289, of SEQ ID NO:2 or SEQ ID NO:6.

33. The isolated nucleic acid molecule of claim 1, wherein the mutated E1A comprises, in consecutive order, amino acids 1 to about 123, amino acids from about 128 to about 188, and amino acids from about 246 to about 289, of SEQ ID NO:2 or SEQ ID NO:6.

34. The isolated nucleic acid molecule of claim 1, wherein the mutated E1A consists of, in consecutive order, amino acids 1 to about 123, amino acids from about 128 to about 188, and amino acids from about 246 to about 289, of SEQ ID NO:2 or SEQ ID NO:6.

35. The isolated nucleic acid molecule of claim 1, wherein the mutated E1A comprises, in consecutive order, amino acids 1 to about 132, and amino acids from about 225-238 of SEQ ID NO:4 or SEQ ID NO:8, wherein amino acids in the CR2 region have been mutated to abolish the ability of the mutated E1A to bind to pRb.

36. The isolated nucleic acid molecule of claim 1, wherein the mutated E1A comprises, in consecutive order, amino acids 1 to about 132, and amino acids from about 200-243 of SEQ ID NO:4 or SEQ ID NO:8, wherein amino acids in the CR2 region have been mutated to abolish the ability of the mutated E1A to bind to pRb.

37. The isolated nucleic acid molecule of claim 1, wherein the mutated E1A comprises, in consecutive order, amino acids 1 to about 123, amino acids from about 128 to about 132, and amino acids from about 225-238 of SEQ ID NO:4 or SEQ ID NO:8.

38. The isolated nucleic acid molecule of claim 1, wherein the mutated E1A comprises, in consecutive order, amino acids 1 to about 123, amino acids from about 128 to about 132, and amino acids from about 200-243 of SEQ ID NO:4 or SEQ ID NO:8.

39. The isolated nucleic acid molecule of claim 1, wherein the mutated E1A consists of in consecutive order, amino acids 1 to about 123, amino acids from about 128 to about 132, and amino acids from about 200-243 of SEQ ID NO:4 or SEQ ID NO:8.

40. The isolated nucleic acid molecule of claim 1, wherein the antigen is selected from the group consisting of a tumor antigen, a viral antigen, a bacterial antigen, a fungal antigen and a parasitic antigen.

41. A recombinant nucleic acid molecule, comprising the isolated nucleic acid molecule of claim 1, operatively linked to at least one transcription control sequence.

42. The recombinant nucleic acid molecule of claim 41, wherein the recombinant nucleic acid molecule comprises a plasmid vector.

43. The recombinant nucleic acid molecule of claim 41, wherein the recombinant nucleic acid molecule comprises a multicistronic vector such that the antigen and mutated E1A protein are expressed as individual proteins.

44. The recombinant nucleic acid molecule of claim 41, wherein the first nucleic acid sequence is operatively linked to the second nucleic acid sequence to produce a fusion protein comprising the antigen linked to the mutated E1A protein.

45. The recombinant nucleic acid molecule of claim 44, wherein the first nucleic acid sequence is operatively linked to the second nucleic acid sequence via a spacer peptide.

46. The recombinant nucleic acid molecule of claim 41, wherein the vector is a viral vector.

47. The recombinant nucleic acid molecule of claim 46, wherein the vector is a recombinant, non-replicating adenoviral vector.

48. The recombinant nucleic acid molecule of claim 47, wherein the first nucleic acid sequence encoding an antigen is cloned into the E3 gene of the adenovirus genome.

49. The recombinant nucleic acid molecule of claim 47, wherein the adenovirus is adenovirus serotype 5 and wherein amino acid positions 124-127 of E1A (SEQ ID NO:2) have been deleted.

50. A fusion protein comprising a mutated E1A protein that is covalently linked to an antigen, wherein the fusion protein is expressed by the recombinant nucleic acid molecule of claim 44.

51. A composition comprising the fusion protein of claim 50.

52. A composition comprising a mutated E1A protein and a tumor antigen, wherein the mutated E1A protein and tumor antigen are expressed by the vector of claim 43.

53. A method to produce a vaccine, comprising:
a) culturing a host cell transfected with a recombinant nucleic acid molecule to produce an antigen and a mutated Group C adenoviral E1A protein, said recombinant nucleic acid molecule comprising:
(i) a first nucleic acid sequence encoding a vaccinating antigen; and
(ii) a second nucleic acid sequence encoding a mutated Group C adenoviral E1A protein;
wherein the amino acid sequence of the mutated E1A protein differs from the amino acid sequence of a wild-type E1A protein by at least one mutation, wherein said at least one mutation comprises a mutation in the CR2 region of E1A that abolishes the ability of the mutated E1A protein to bind to pRb;
wherein the mutated E1A protein retains an amino acid sequence from the wild-type E1A protein that is sufficient to bind to p300/CBP; and
wherein the mutated E1A protein retains an amino acid sequence from the wild-type E1A protein that binds to the C-terminal binding protein (CtBP); and
b) recovering the antigen and mutated Group C adenoviral E1A protein from the host cell.

54. The method of claim 53, wherein the recombinant nucleic acid molecule comprises a multicistronic vector so that the antigen and mutated E1A protein are expressed as individual proteins.

55. The method of claim 53, wherein the first nucleic acid sequence encoding the antigen is operatively linked to the second nucleic acid sequence encoding a mutated E1A protein to produce a fusion protein comprising the antigen linked to the mutated E1A protein.

56. The method of claim 55, wherein the antigen and mutated E1A protein are linked via an intervening spacer peptide.

57. A recombinant modified, non-transforming Group C adenovirus, wherein the genome of the modified adenovirus differs from the genome of a wild-type Group C adenovirus by at least the following modifications:
a) a mutation in the adenoviral genome that renders the recombinant adenovirus non-transforming;
b) a mutation in the genome in the region encoding amino acids of the E1A CR2 domain that is sufficient to abolish binding of the E1A protein to pRb; and
c) an insertion of a nucleic acid sequence encoding an antigen into the E3 region of the genome;
wherein the modified adenovirus retains a portion of the E1 genome that encodes an amino acid sequence from the wild-type E1A protein that is sufficient to bind to p300/CBP; and
wherein the modified adenovirus retains a portion of the E1 genome that encodes an amino acid sequence from the wild-type E1A protein that binds to the C-terminal binding protein (CtBP).

58. The recombinant adenovirus of claim 57, wherein the wild-type adenovirus is selected from the group consisting of adenovirus serotype 2 and adenovirus serotype 5.

59. The recombinant adenovirus of claim 57, wherein a portion of the genome encoding amino acids 124-127 of E1A (SEQ ID NO:2 or SEQ ID NO:6) is deleted.

60. The recombinant adenovirus of claim 57, wherein the recombinant modified adenovirus has an E1A protein comprising an amino acid sequence that differs from the amino acid sequence of the wild-type E1A by a deletion of amino acids in the CR2 region that abolishes the ability of the mutated E1A protein to bind to pRb.

61. The recombinant adenovirus of claim 57, wherein the recombinant modified adenovirus has an E1A protein comprising an amino acid sequence that differs from the amino acid sequence of the wild-type E1A by a substitution or insertion of amino acids in the CR2 region that abolishes the ability of the mutated E1A protein to bind to pRb.

62. The recombinant adenovirus of claim 57, wherein the recombinant modified adenovirus has an E1A protein comprising an amino acid sequence that is identical to amino acid sequence of the wild-type E1A by retention of amino acid residues in the first 60 amino acid residues of the N-terminus of the protein that are sufficient to bind to p300/CBP.

63. A method for eliciting an antigen specific immune response in a patient, comprising administering to the patient the nucleic acid molecule of claim 1.

64. A method for eliciting an antigen specific immune response in a patient, comprising administering to the patient the fusion protein of claim 50.

65. A method of eliciting an antigen specific immune response in a patient, comprising administering to the patient the composition of claim 52.

66. A method of eliciting an antigen specific immune response in a patient, comprising administering to the patient the recombinant adenovirus of claim 57.

67. A method for treating a patient that has a tumor, comprising administering to the patient the nucleic acid molecule of claim 1, wherein the antigen is a tumor antigen.

68. A method for treating a patient that has a tumor, comprising administering to the patient the fusion protein of claim 50, wherein the antigen is a tumor antigen.

69. A method for treating a patient that has a tumor, comprising administering to the patient the composition of claim 52, wherein the antigen is a tumor antigen.

70. A method for treating a patient that has a tumor, comprising administering to the patient the recombinant adenovirus of claim 57, wherein the antigen is a tumor antigen.


SEQUENCE LISTING (8 sequences)

SEQ ID NO: 1 - DNA, 870 bp, Human adenovirus type 5
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 2 - protein, 289 aa, Human adenovirus type 5
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 3 - DNA, 732 bp, Human adenovirus type 5
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataa

SEQ ID NO: 4 - protein, 243 aa, Human adenovirus type 5
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 5 - DNA, 870 bp, Human adenovirus type 2
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataa

SEQ ID NO: 6 - protein, 289 aa, Human adenovirus type 2
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 7 - DNA, 732 bp, Human adenovirus type 2
atgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataa

SEQ ID NO: 8 - protein, 243 aa, Human adenovirus type 2
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP